(19) United States
(12) Patent Application Publication
(10) Pub. No.: US 2024/0185965 A1
(43) Pub. Date: Jun. 6, 2024

(54) Computational Model and Methods for Selecting Clinical Trial Subjects to Reduce Heterogeneity

(71) Applicant: SUNOVION PHARMACEUTICALS INC., Marlborough, MA (US)

(72) Inventors: Seth Cabot HOPKINS, Marlborough, MA (US); Sasagu TOMIOKA, Marlborough, MA (US)

(21) Appl. No.: 18/284,130

(22) PCT Filed: Mar. 30, 2022
(86) PCT No.: PCT/US2022/071447

§ 371 Date: Sep. 26, 2023

Related U.S. Application Data
(60) Provisional application No. 63/226,595, filed on Jul. 28, 2021.
(60) Provisional application No. 63/168,685, filed on Mar. 31, 2021.

(51) Int. Cl. G16H 10/20; G16H 20/70; G16H 50/20

(52) U.S. Cl. CPC G16H 10/20; G16H 20/70; G16H 50/20

(57) ABSTRACT

Clinical study populations require reduced heterogeneity to properly determine effectiveness of treatments. In an embodiment, a method of verifying eligibility of a subject for a treatment includes representing the subject's symptoms in a rating scale as a vector. The method computes an anomaly score based on the vector of the subject and multiple vectors representing rating scales of other subjects. The method ranks, based on the anomaly score, the subject with a likelihood of contributing to a subgroup of patients having a common element structure of the rating scale. The method enriches a study population in a clinical trial prior to randomization, the enriched study population having a reduced heterogeneity. Therefore, the method can verify diseases or conditions or diagnoses of subjects for eligibility for a clinical trial or for other purposes.

### RELATED APPLICATIONS

This application claims the benefit of U.S. Provisional Application No. 63/168,685, filed on Mar. 31, 2021, and U.S. Provisional Application No. 63/226,595, filed on Jul. 28, 2021. The entire teachings of the above applications are incorporated herein by reference.

### FIELD

Provided herein are methods, including methods of enriching a population, and/or methods of identifying subjects that conform to the expected symptom presentation, and/or methods of reducing heterogeneity, and/or methods of rejecting anomalous subjects, for example, for a clinical trial.

### BACKGROUND

Conducting a clinical trial (e.g., investigating the safety and efficacy of an active pharmaceutical ingredient) requires significant resources and planning, including identifying an appropriate prospective patient population. If the population has subjects who do not have an appropriate diagnosis, the results of the trial may be distorted. For example, a subject who does not have an appropriate diagnosis, but is included in the study and receives the treatment, may distort the results such that the proportion of subjects that responded effectively to the treatment is skewed. As another example, in some clinical trials for drugs for treating negative symptoms in schizophrenia, patients can be selected based on severity and stability of negative symptoms relative to positive symptoms using criteria which are not suitable for trials of acute exacerbation of schizophrenia.

Methods for mitigating the risk of skewed results include taking an otherwise eligible subject from a pool of prospective subjects and (a) verifying each individual subject's diagnosis, or (b) verifying ongoing symptoms to ensure consistency with canonical presentation of disease or disorder, which are sometimes called “consistency checks.” Either of these methods can include using tools like medical records to further support including a subject in a clinical trial. However, despite these methods, subjectivity in diagnosis and medical records remains such that a subject may appear to have a particular diagnosis, but is otherwise unfit to receive treatment.

### SUMMARY

Accordingly, there is a need for a method of enriching a population, and/or identifying subjects that conform to the expected symptom presentation, and/or reducing heterogeneity, and/or rejecting anomalous subjects, for example, for a clinical trial.

In some embodiments, provided herein is a method of verifying eligibility of a subject for a treatment comprising representing the subject's symptoms in a rating scale as a vector, computing an anomaly score based on the vector of the subject, and multiple vectors representing rating scales of symptoms of other subjects. In some embodiments, the method includes, based on the anomaly score, ranking the subject with a likelihood of contributing to a subgroup of patients having a common element structure of the rating scale. In some embodiments, the method includes enriching a study population in a clinical trial prior to randomization, such that the enriched study population has a reduced heterogeneity.

In some embodiments, provided is a method of verifying eligibility of a subject for a treatment comprising administering a test, to the subject, that measures multiple symptoms of the subject. In some embodiments, the method includes computing an anomaly score based on a comparison of each element of the test administered to a respective expected pattern. In some embodiments, the method includes computing an anomaly score based on how far the test administered is from the expected test administered considering multiple elements. In some embodiments, the method includes computing an anomaly score based on the number of steps necessary to isolate a subject from other subjects. In some embodiments, the method includes, based on the anomaly score, assigning, to the subject, a likelihood of having a condition related to the treatment.

In some embodiments, provided is a method of improving a clinical dataset, wherein the method includes, for a subject of the clinical dataset, computing an anomaly score based on a comparison of multiple elements of a diagnosis test to respective expected patterns. In some embodiments, provided is a method of improving a clinical dataset, wherein the method includes, for a subject of the clinical dataset, computing an anomaly score based on a comparison of the structure of psychiatric elements of the test administered to a respective expected structure. In some embodiments, the method includes, if the anomaly score of the subject is above a particular threshold, removing data corresponding to the subject from the clinical dataset, or if the anomaly score of the subject is below the particular threshold, including the data corresponding to the subject from the clinical dataset.

In some embodiments, provided is a method of verifying eligibility of a subject for a treatment of depression, wherein the method includes administering a test, to the subject, that measures multiple psychiatric elements of the subject, computing an anomaly score based on a comparison of each psychiatric element of the test administered to a respective expected pattern, and based on the anomaly score, assigning a likelihood of having depression to the subject.

In some embodiments, provided is a method of verifying a diagnosis of a subject, wherein the method includes administering a test, to the subject, that measures multiple symptoms of the subject. In some embodiments, the method includes computing an anomaly score based on a comparison of each element of the multiple elements of the test administered to a respective expected pattern and, based on the anomaly score, assigning a likelihood of having a condition related to the treatment to the subject. In some embodiments, the method includes computing an anomaly score based on how far the test administered is from the expected test administered considering multiple elements and, based on the anomaly score, assigning a likelihood of having a condition related to the treatment to the subject.

In some embodiments, provided is a method of treating a subject having a psychiatric condition, wherein the method includes administering to the subject a therapeutically effective amount of a treatment for the condition. Prior to receiving the treatment, the subject is determined to be eligible for the treatment by administering a test, to the subject, that measures multiple symptoms of the subject, computing an anomaly score based on a comparison of each element of the multiple elements of the test administered to a respective expected pattern, and based on the anomaly score, assigning to the subject a likelihood of having the psychiatric condition.

In some embodiments, provided is a method of treating a subject having a psychiatric condition, wherein the method includes administering to the subject a therapeutically effective amount of a treatment for the condition. Prior to receiving the treatment, the subject is determined to be eligible for the treatment by administering a test, to the subject, that measures multiple symptoms of the subject, computing an anomaly score based on a trained forest model, and based on the anomaly score, assigning to the subject a likelihood of having the psychiatric condition.

In some embodiments, provided is a method of treating a subject having bipolar I depression, wherein the method includes administering to the subject a therapeutically effective amount of a therapeutic agent. In some embodiments, the method includes, prior to receiving the therapeutically effective amount, the subject is determined to be eligible for a treatment comprising a therapeutic agent by administering a Montgomery-Åsberg Depression Rating Scale (MADRS) test, to the subject, that measures multiple symptoms of the subject, computing an anomaly score based on a comparison of each element of the multiple elements of the MADRS test administered to a respective expected pattern, and based on the anomaly score, assigning a likelihood of having bipolar I depression to the subject. In some embodiments, the method includes, prior to receiving the therapeutically effective amount, the subject is determined to be eligible for a treatment comprising a therapeutic agent by administering a Montgomery-Åsberg Depression Rating Scale (MADRS) test, to the subject, that measures multiple symptoms of the subject, computing an anomaly score based on how far the MADRS test administered is from the expected test administered considering multiple elements, and based on the anomaly score, assigning a likelihood of having bipolar I depression to the subject.

In some embodiments, provided is a method of verifying treatment eligibility of a subject exhibiting one or more symptoms characterized in a rating scale, wherein the method includes characterizing the subject's symptoms in the rating scale as a subject vector and characterizing a one or more of other subjects' symptoms in the rating scale as multiple population vectors. Each population vector corresponds with one of the other subjects. In some embodiments, the method includes computing an anomaly score based on the subject vector and the population vectors, and, based on the anomaly score, verifying treatment eligibility of the subject by ranking the subject with a likelihood of contributing to a sub-population of subjects having a common element structure of the rating scale.

In some embodiments, a method for determining subject participation in a clinical trial includes receiving one or more rater inputs reflecting the rater's clinical evaluation of a severity of a previously diagnosed condition in a subject. In some embodiments, the method includes performing a computerized assessment of the subject to quantify severity of the previously diagnosed condition in the subject through a computerized interview that comprises presenting a plurality of questions to the subject and receiving a plurality of corresponding inputs from the subject in response thereto, based on plurality of inputs received from the subject, determining an anomaly score for the condition in the subject, and determining, via a processor, a recommendation of including or excluding the subject from the clinical trial.

In some embodiments, a computer-implemented method of identifying one or more clinical study candidates, includes receiving consolidated health care information for a consumer of medical services. In some embodiments, the method includes retrieving, by one or more computers, attributes defining a suitable candidate for a clinical study, the attributes based on rating data of patients tested for a condition. In some embodiments, the method includes causing the one or more computers to compare the attributes defining the suitable candidate for the clinical study to the consolidated health information for the consumer. In some embodiments, the method includes determining by the one or more computers that the consumer's consolidated health information includes at least one of the attributes defining the suitable candidate for the clinical study. In some embodiments, the method includes identifying the consumer as eligible to participate in the clinical study. In some embodiments, the method includes notifying an administrator of the clinical study that the consumer is eligible to participate in the clinical study.

Provided herein is a method to enrich for subjects having a specific predefined factor structure in the Positive and Negative Syndrome Scale (PANSS), which is applicable to any trial population. Also provided herein is a vector of 1335 elements based on between- and within-item variance, covariance and differences of PANSS items used to calculate an index of heterogeneity for each of any predetermined symptom construct in PANSS. Using pre-randomization data, enrichment is demonstrated for subjects who maintain the maximal variance explained by the 7-item of the Marder PANSS negative symptom (MPNS) construct that is robust and generalizable across N=4,876 subjects in 13 trials of acute schizophrenia. These results demonstrate that psychometric properties of PANSS derived from factor analyses in large sample sizes, can be used at the individual subject level and applied towards prognostic enrichment of clinical trials.

In some embodiments, provided is a method of treating a subject having schizophrenia, wherein the method includes administering to the subject a therapeutically effective amount of a therapeutic agent. In some embodiments, the method includes, prior to receiving the therapeutically effective amount, the subject is determined to be eligible for a treatment comprising a therapeutic agent by administering a Positive and Negative Syndrome Scale (PANSS) test, to the subject, that measures multiple symptoms of the subject, computing an anomaly score based on a comparison of each element of the multiple elements of the PANSS test administered to a respective expected pattern, and based on the anomaly score, assigning a likelihood of having schizophrenia to the subject. In some embodiments, the method includes, prior to receiving the therapeutically effective amount, the subject is determined to be eligible for a treatment comprising a therapeutic agent by administering a PANSS test, to the subject, that measures multiple symptoms of the subject, computing an anomaly score based on how far the PANSS test administered is from the expected test administered considering multiple elements, and based on the anomaly score, assigning a likelihood of having schizophrenia to the subject.

A person having ordinary skill in the art can understand that the methods above are not limited and can have variations and permutations as described herein and as are known in the art. A person having ordinary skill in the art can also understand that the methods described above can further be implemented as a system that includes hardware (e.g., including a processor and memory). A person having ordinary skill in the art can further understand that the above methods can be implemented by a processor and memory having instructions of any of the above methods stored thereupon, such that when the processor executes the instructions in the memory, it executes the steps of the respective method.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-E are block diagrams illustrating example embodiments of the present disclosure.

FIG. 2 is a flow diagram illustrating example embodiments of a method of the present disclosure.

FIG. 3 is a diagram illustrating example embodiments of the present disclosure.

FIG. 4 is a block diagram illustrating an example embodiment of the present disclosure.

FIG. 5 is a block diagram illustrating an example embodiment of the present disclosure.

FIG. 6 is a graph 700 illustrating a threshold for enriching subjects for having a Marder PANSS Negative Symptom (MPNS) construct.

FIG. 7 are graphs 800 illustrating variances explained based on various factors on the PANSS scale.

FIG. 8 are graphs 900 illustrating factor scores for a study of the drug ulotaront. Graph 902 illustrates the Marder Negative PANSS Symptom Factor Score (MPNS) for all subjects as measured at each week during a double-blind baseline study for 125 placebo patients and 120 patients being administered ulotaront.

FIG. 9 are graphs 1000 illustrating factor scores for a study of the drug lurasidone. Graph 1002 illustrates the Marder Negative PANSS Symptom Factor Score (MPNS) for all subjects as measured at each week during a double-blind baseline study for 504 placebo patients and 1041 patients being administered lurasidone.

FIG. 10A is a diagram 1050 illustrating an example of the MADRS anomaly detector's inclusion criteria.

FIG. 10B is a diagram 1070 illustrating an example of the PANSS Negative Heterogeneity Detector.

FIG. 11A is a diagram 1100 illustrating a graph of subjects enriched for having MPNS constructs.

FIG. 11B is a diagram 1150 illustrating a graph of subjects de-enriched for having MPNS constructs.

FIG. 12 is a diagram illustrating a computer network or similar digital processing environment in which embodiments of the present invention may be implemented.

FIG. 13 is a diagram of an example internal structure of a computer (e.g., client processor/device or server computers) in the computer system of FIG. 12.

### DETAILED DESCRIPTION

Provided herein are methods, including methods of enriching a population, and/or methods of identifying subjects that conform to the expected symptom presentation, and/or methods of reducing heterogeneity, and/or methods of rejecting anomalous subjects. Such methods may improve a signal of effectiveness of a drug or treatment (e.g., reduce noise) in a clinical trial or study by screening patients who are objectively exhibiting symptoms or elements that are contrary to the element structure. For example, if patients that exhibited anomalies of having a condition are screened out, the separation between placebo and drug is more recognizable because there is a more accurate population of the people having the disease or condition being studied in clinical trial or study. By removing anomalous patients, the drug effect is more accurately measured because the drug or placebo is not given to an individual who does not actually have the disease or condition the study is testing against. For example, the present disclosure reduces the likelihood that a person who does not have a bipolar condition that somehow makes it through screening, and therefore exhibits a greater placebo effect than expected in a patient who has the bipolar condition.

In current methods, inclusion criteria for clinical trials use severity of factor based tests. However, using severity of factors alone ignores the structure of all of the factors together, and also ignores negative symptoms. In embodiments, methods use structure of factor based tests to determine inclusion criteria, which is insensitive to changes in severity of symptoms. After randomization of populations, factor structure is a more stable inclusion criteria than factor severity.

The FDA has outlined three categories of enrichment strategies for clinical trials in a March 2019 document entitled “Enrichment Strategies for Clinical Trials to Support Determination of Effectiveness of Human Drugs and Biological Products Guidance for Industry”:
(1) Strategies to decrease variability—These strategies include choosing patients with baseline measurements of a disease or a biomarker characterizing the disease in a narrow range (decreased interpatient variability) and excluding patients whose disease or symptoms improve spontaneously or whose measurements are highly variable (decreased intrapatient variability). The decreased variability provided by these strategies would increase study power (see section III., Decreasing Variability).
(2) Prognostic enrichment strategies—These include choosing patients with a greater likelihood of having a disease-related endpoint event (for event-driven studies) or a substantial worsening in condition (for continuous measurement endpoints) (see section IV., Prognostic Enrichment Strategies—Identifying High-Risk Patients). These strategies would increase the absolute effect difference between groups but would not be expected to alter relative effect.
(3) Predictive enrichment strategies—These include choosing patients who are more likely to respond to the drug treatment than other patients with the condition being treated. Such selection can lead to a larger effect size (both absolute and relative) and can permit use of a smaller study population. Selection of patients could be based on a specific aspect of a patient's physiology, a biomarker, or a disease characteristic that is related in some manner to the study drug's mechanism. Patient selection could also be empiric (e.g., the patient has previously appeared to respond to a drug in the same class) (see section V., Predictive Enrichment—Identifying More-Responsive Patients).

In a first embodiment, an anomaly detector method reduces heterogeneity relative to information (e.g., MADRS scores) collected in clinical trials or other datasets. In a second embodiment, an enrichment method pre-defines a desired factor structure (e.g., of a PANSS score) and enriches the population for that desired factor structure one subject at a time. In other words, this embodiment captures properties of PANSS at the subject-level, rather than needing a population at larger N for properties to emerge. While both the first and second embodiments employ variance-covariance-difference vector formalism, the first embodiment employs a machine learning method on that vector.

The anomaly detector method of the first embodiment is considered a strategy to decrease variability (i.e., FDA strategy 1). The enrichment method of the second embodiment is considered a prognostic enrichment strategy (i.e., FDA strategy 2).

### Definitions

As used herein, and unless otherwise specified, the following definitions inform the application throughout.

An anomaly score is one or more deviations of presentation of a subject's test results from expected presentation for a condition. For example, an anomaly score is an indicator of how different the response patterns are from the expected response patterns.

An element is a subject's response to a test or diagnostic test that is a measure of the subject's physical or mental state. The subject's response can be an answer to a question, such as rating symptoms on a numerical scale or a yes or no question (e.g., a binary element). The subject's response, in other embodiments, can be a physical response measured in an objective manner. Elements can include data such as a rating scale, latent variables, or domains of a rating scale, but can also include demographic information in some embodiments. In some embodiments, elements can be items of a MADRS or PANSS score, which are described further below. In some embodiments, elements can be inferred by the items of a MADRS or PANSS score, which are described further below. In some embodiments, an element is an unobserved variable that can be inferred from set of subject's responses.

The Montgomery-Åsberg Depression Rating Scale (MADRS) score is a ten-item mood disorder diagnostic questionnaire, wherein each item is scored by a professional (e.g., a psychiatrist) on a scale of 0-6 for a total score of 0-60, with a higher score implies a worse symptom. The ten MADRS items are: 1. Apparent sadness; 2. Reported sadness; 3. Inner tension; 4. Reduced sleep; 5. Reduced appetite; 6. Concentration difficulties; 7. Lassitude; 8. Inability to feel; 9. Pessimistic thoughts; and 10. Suicidal thoughts. In general, many of the MADRS items are either highly correlated or highly inversely correlated. One example of a pair of highly correlated items is “apparent sadness” and “reported sadness.” A person who scores high on “apparent sadness” is expected to also score high on “reported sadness” because those items are highly related. In other words, a person who appears sad should report sadness. From “apparent sadness” and “reported sadness”, a latent element “sadness” can be inferred.

The Positive and Negative Syndrome Scale (PANSS) is a 30-item schizophrenia severity diagnostic questionnaire, where each item is scored by a professional (e.g., a psychiatrist) on a scale of 1-7 for a total score of 30-210. The PANSS items are categorized within a positive scale (7 items), a negative scale (7 items), and a general psychopathology scale (16 items). The positive scale items are: 1. Delusions; 2. Conceptual disorganization; 3. Hallucinations; 4. Excitement; 5. Grandiosity; 6. Suspiciousness/persecution; 7. Hostility. The negative scale items are 1. Blunted affect; 2. Emotional withdrawal; 3. Poor rapport; 4. Passive/apathetic social withdrawal; 5. Difficulty in abstract thinking; 6. Lack of spontaneity and flow of conversation; 6. Stereotyped thinking. The general psychopathology scale items are Somatic concern; 2. Anxiety; 3. Guilt feelings; 4. Tension; 5. Mannerisms and posturing; 6. Depression; 7. Motor retardation; 8. Uncooperativeness; 9. Unusual thought content; 10. Disorientation; 11. Poor attention; 12. Lack of judgment and insight; 13. Disturbance of volition; 14. Poor impulse control; 15. Preoccupation; and 16. Active social avoidance.

A psychiatric element is an element solely related to the subject's mental state.

An element structure is a mathematical description of variability, where correlated variables in a rating scale's item scores are described by a lower number of latent variables (e.g., elements).

A vector is a one-dimensional array of a plurality of values derived from the item scores in a rating scale for a single subject. A subject vector can be an array of elements relating to a subject.

A therapeutic agent includes any chemical or biological substance or material for treating a human exhibiting a disease, disorder, condition, symptom or the like. A person having ordinary skill in the art can recognize that therapeutic agents can be exploratory (e.g., under investigation in clinical trials) or commercial (e.g., approved by a regulatory agency after a showing of safety and/or efficacy). The United States Food and Drug Administration (FDA) catalogs all approved therapeutic agents as well as those undergoing clinical trials in publicly accessible databases. As used herein, a therapeutic agent may be any one or more, in combination or individually, of the therapeutic agents in the FDA's databases.

A treatment includes administering a drug or therapy to a patient (e.g., in a clinical trial, or other study) and/or determining whether to administer a drug or therapy to a patient in a clinical trial, or other study. A person having ordinary skill in the art can recognize that, in addition to enriching a study population, the methods disclosed herein can be used to verify a diagnosis of a subject outside of a study or trial context.

Provided herein are non-limiting exemplary embodiments intended to describe the disclosed invention(s), and/or aspects thereof. Where context permits, embodiments can be combined with any other embodiment(s) disclosed herein or disclosed in any reference incorporated herein. While example embodiments are shown and described in this disclosure, it will be understood by those skilled in the art that various changes in form and details may be made therein without departing from the scope of the embodiments encompassed by the appended claims. The embodiments described herein are non-limiting and can be combined with other embodiments, including embodiments that are incorporated by reference by this disclosure.

In some embodiments, provided is a method and/or computational model that enriches clinical trial study populations to reduce the heterogeneity of the study population. In some embodiments, provided is a method of identifying subject(s) of a clinical trial that conform to the expected symptom presentation of an expected study population. In some embodiments, provided is a method for reducing statistical noise within the study population by, for example, detecting and removing anomalous subjects. In some embodiments, provided is a method of screening a study population (e.g., for a bipolar depression clinical trial), wherein the method determines whether a patient exhibits an anomaly in their Montgomery-Åsberg Depression Rating Scale (MADRS) score. In some embodiments, provided is a method of screening or pre-screening patients for a treatment, clinical trial, or clinical study (e.g., screen out patients who should not respond to the medication treating the disease or condition). In some embodiments of the present disclosure, provided is a method that can be used to verify eligibility of a patient for a treatment, where such verification can be used for enriching a population for a treatment, identifying subjects that conform to the expected symptom presentation of a test, reducing heterogeneity of a population for a treatment, and rejecting anomalous subjects for a treatment.

In some embodiments, provided is a method for pre-screening a disease or condition based on MADRS. In some embodiments, provided is a method for pre-screening diseases or conditions based on other item based tests, such as Positive and Negative Syndrome Scale (PANSS). For example, in some embodiments, a method is provided that allows a subject into the study when the subject has a qualifying score on the screening test (e.g., MADRS score, PANSS score, etc.) and not having an anomaly within the elements of the screening test. Consequently, in some embodiments the method excludes subjects from the study when the subject has a qualifying score of the screening test, but does have an anomaly within the response patterns of the screening test.

In some embodiments, a method of verifying eligibility of a subject for a treatment includes representing the subject's symptoms in a rating scale as a vector, and computing an anomaly score based on the vector of the subject and multiple vectors representing rating scales of symptoms of other subjects. In some embodiments, the method includes, based on the anomaly score, ranking the subject with a likelihood of contributing to a subgroup of patients having a common element structure of the rating scale. In some embodiments, the method includes enriching a study population in a clinical trial prior to randomization, such that the enriched study population has a reduced heterogeneity.

In some embodiments, a method of verifying eligibility of a subject for a treatment includes administering a test, to the subject, that measures multiple symptoms of the subject. In some embodiments, the method includes computing an anomaly score based on a comparison of each element of the test administered to a respective expected pattern. In some embodiments, the method includes, based on the anomaly score, assigning, to the subject, a likelihood of having a condition related to the treatment.

In some embodiments, a method of improving a clinical dataset includes, for a subject of the clinical dataset, computing an anomaly score based on a comparison of multiple elements of a diagnosis test to respective expected patterns. In some embodiments, the method includes, if the anomaly score of the subject is above a particular threshold, removing data corresponding to the subject from the clinical dataset, or if the anomaly score of the subject is below the particular threshold, including the data corresponding to the subject from the clinical dataset.

In some embodiments, a method of verifying eligibility of a subject for a treatment of depression includes administering a test, to the subject, that measures multiple psychiatric symptoms of the subject, computing an anomaly score based on a comparison of each psychiatric element of the test administered to a respective expected pattern, and based on the anomaly score, assigning a likelihood of having depression to the subject.

In some embodiments, a method of verifying a diagnosis of a subject includes administering a test, to the subject, that measures multiple symptoms of the subject. In some embodiments, the method includes computing an anomaly score based on a comparison of each element of the multiple elements of the test administered to a respective expected pattern and, based on the anomaly score, assigning a likelihood of having a condition related to the treatment to the subject.

In some embodiments, a method of treating a subject having a psychiatric condition includes administering to the subject a therapeutically effective amount of a treatment for the condition. Prior to receiving the treatment, the subject is determined to be eligible for the treatment by administering a test, to the subject, that measures multiple symptoms of the subject, computing an anomaly score based on a comparison of each element of the multiple elements of the test administered to a respective expected pattern, and based on the anomaly score, assigning to the subject a likelihood of having the psychiatric condition.

In some embodiments, a method of treating a subject having bipolar I depression includes administering to the subject a therapeutically effective amount of a therapeutic agent. In some embodiments, the method includes, prior to receiving the therapeutically effective amount, the subject is determined to be eligible for a treatment comprising a therapeutic agent by administering a Montgomery-Åsberg Depression Rating Scale (MADRS) test, to the subject, that measures multiple symptoms of the subject, computing an anomaly score based on a comparison of each element of the multiple elements of the MADRS test administered to a respective expected pattern, and based on the anomaly score, assigning a likelihood of having bipolar I depression to the subject. In some embodiments, the method includes, prior to receiving the therapeutically effective amount, the subject is determined to be eligible for a treatment comprising a therapeutic agent by administering a Montgomery-Åsberg Depression Rating Scale (MADRS) test, to the subject, that measures multiple symptoms of the subject, computing an anomaly score based on how far the MADRS test administered is from the expected test administered considering multiple elements, and based on the anomaly score, assigning a likelihood of having bipolar I depression to the subject.

In some embodiments, a method of verifying treatment eligibility of a subject exhibiting one or more symptoms characterized in a rating scale includes characterizing the subject's symptoms in the rating scale as a subject vector and characterizing a one or more of other subjects' symptoms in the rating scale as multiple population vectors. Each population vector corresponds with one of the other subjects. In some embodiments, the method includes computing an anomaly score based on the subject vector and the population vectors, and, based on the anomaly score, verifying treatment eligibility of the subject by ranking the subject with a likelihood of contributing to a sub-population of subjects having a common element structure of the rating scale.

In some embodiments, the anomaly score is based on determining a path length from a starting node to an ending node of a binary tree grown from the vector of the subject and the plurality of vectors of the other subjects.

In some embodiments, the starting node is a root node and the ending node is a terminal external node.

In some embodiments, the binary tree is an isolation forest. In some embodiments, the binary tree is an element of an isolation forest.

In some embodiments, improving a clinical dataset by employing the enriched study population.

In some embodiments, enriching the study population includes verifying a diagnosis of the subject based on the anomaly score.

In some embodiments, enriching the study population includes identifying the subject as an outlier compared to the subgroup of patients based on the anomaly score.

In some embodiments, the multiple symptoms are of a disease or condition. In some embodiments, the method includes treating the subject having the disease or condition based on the anomaly score.

In some embodiments, the test is a diagnostic test that measures a subjective condition of the subject.

In embodiments, the test is a diagnostic test that measures depression of the subject.

In some embodiments, the subjective condition is one or more of bipolar disorder, depression, and schizophrenia.

In some embodiments, the test is at least one of the Montgomery-Åsberg Depression Rating Scale (MADRS) and Positive and Negative Syndrome Scale (PANSS). In some embodiments, other tests are employed.

In some embodiments, the method includes administering the test, to the subject, includes administering the test at multiple time points and computing the anomaly score compares elements at different time points for the subject.

In some embodiments, the method includes, based on the likelihood of having the condition, determining the subject's eligibility for receiving the treatment.

In some embodiments, the method includes, based on the likelihood of having the condition, determining the subject's eligibility for a clinical trial of the treatment.

In some embodiments, the method includes, (a) if the likelihood of the subject having the condition is below a threshold, marking the subject as a candidate for removal from a clinical trial of the treatment, or (b) otherwise, marking the subject as a candidate for the clinical trial of the treatment.

In some embodiments, the method includes, if the likelihood of the subject having the condition is above a threshold, marking the subject as a candidate for a clinical trial of the treatment.

In some embodiments, computing the anomaly score is performed by weighting the pattern of each element to each other element in accordance to its respective expected pattern.

In some embodiments, the method includes computing deviance from expected pattern for each element compared to all other elements, and calculating the anomaly scored based on the deviance from expected pattern.

In some embodiments, elements are subjective elements.

In some embodiments, the method includes improving a clinical dataset by either including or excluding the subject from the clinical dataset, the inclusion or exclusion based on the assigned likelihood of the subject having the condition.

In some embodiments, the method includes verifying a diagnosis of the subject based on the anomaly score.

In some embodiments, the method includes identifying the subject as an outlier compared to the respective expected pattern based on the anomaly score.

In some embodiments, the multiple elements are of a disease or condition. The method includes treating the subject having the disease or condition based on the anomaly score.

In some embodiments, the method includes verifying a diagnosis of bipolar I depression of the subject based on the anomaly score.

In some embodiments, provided is a method that includes automatically notifying a designer of a study of subjects qualifying for the study and subjects to be removed from the study.

In some embodiments, provided is a method that automatically determines whether subjects qualify for a study, and further automatically filters data from subjects not qualifying for the study.

In some embodiments, provided is a method that performs statistical analysis of a study population having been enriched by the above methods, the statistical analysis performed by automatically removing anomalous subjects from the study population, free of notification of the study designer. Therefore, if the study designer desires, the study designer can be blind to the enrichments to the population performed by the method.

In some embodiments, the method is implemented by a processor and a memory having instructions for any of the above embodiments thereon, the processor configured to execute said instructions.

The above embodiments may demonstrate several improvements in clinical trials and diagnosis of conditions. The above methods of the above embodiments may display consistent improvement in treatment effect after reducing the noise of study populations. Significantly, the subjects that introduce noise to the study outcome can be detected using pre-randomization data, which increases the success of the clinical trials by screening/not allowing that subject to enroll. In embodiments, the above methods can be applied for other diseases or conditions (e.g., schizophrenia, ADHD) where the primary endpoint includes subjective measures. As used herein, a disease or condition can be a diagnosis or a likelihood that a subject has a condition/disorder.

In some embodiments, the present disclosure provides methods that can solve multiple problems associated with verifying a diagnosis of a patient and pre-screening or screening patients for clinical trials, clinical studies, or treatments. First, patients may believe they are diagnosed with a condition that they never had, or no longer have. The methods of the present disclosure are useful for identifying such patients and preventing their admission into said trials, studies, or treatments. Second, candidates may attempt to join a study for compensation or other reasons without having a required condition. The methods of the present disclosure are useful for identifying such people who do not have the condition, and excluding them from the study population.

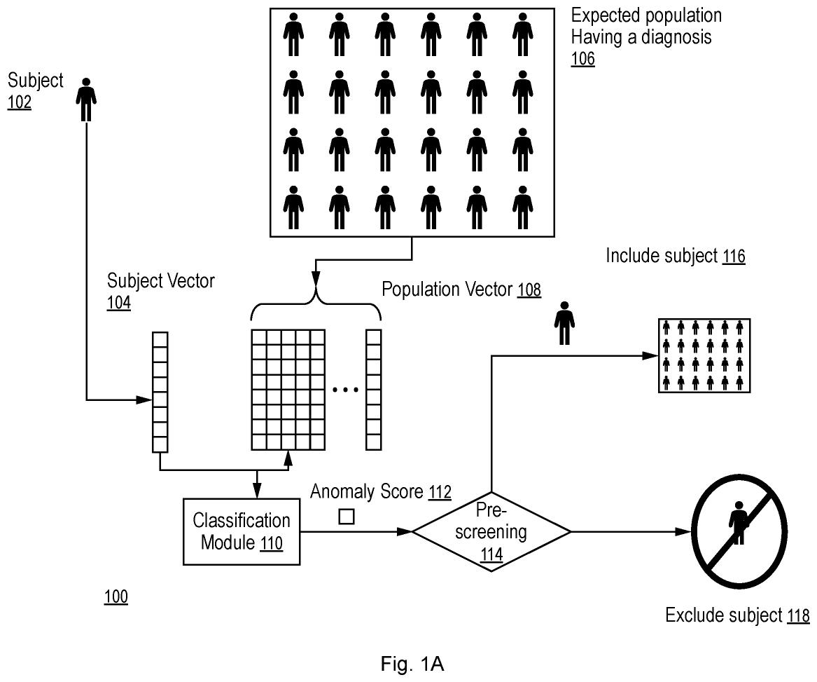

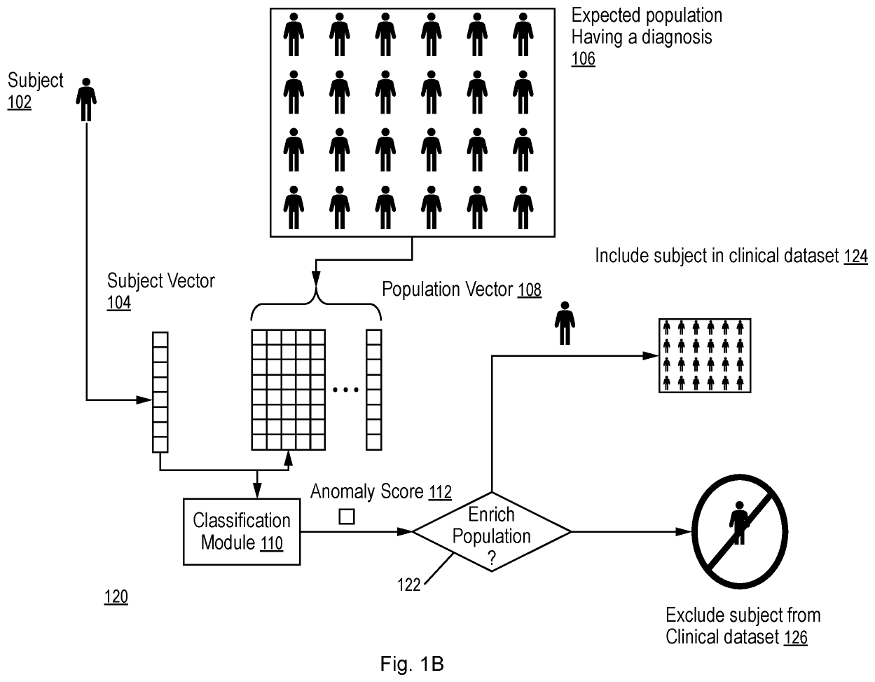

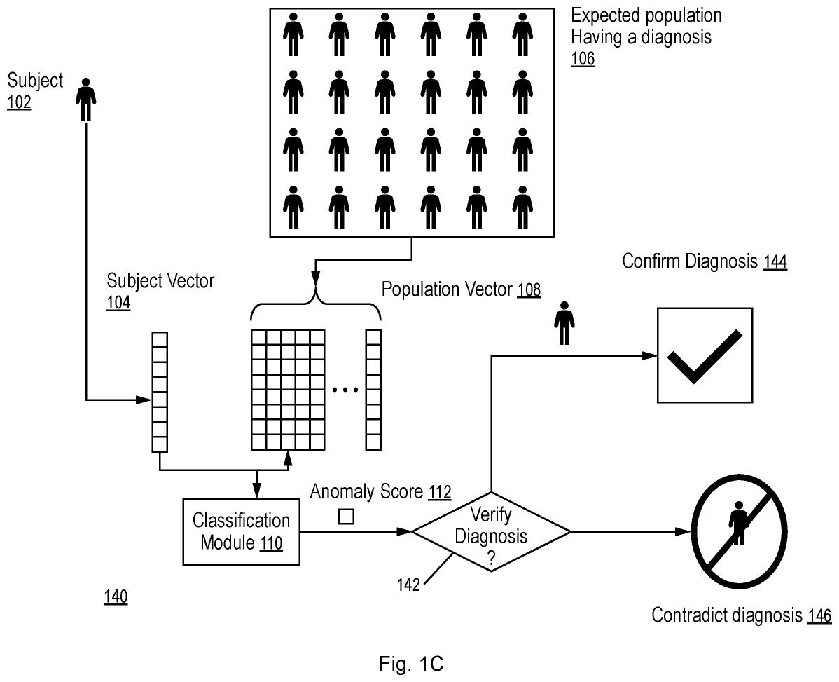

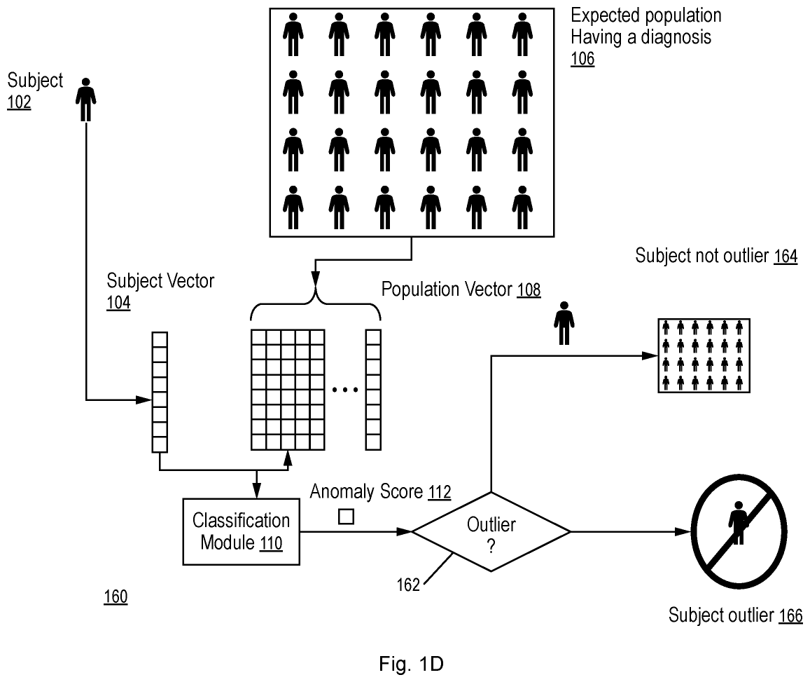

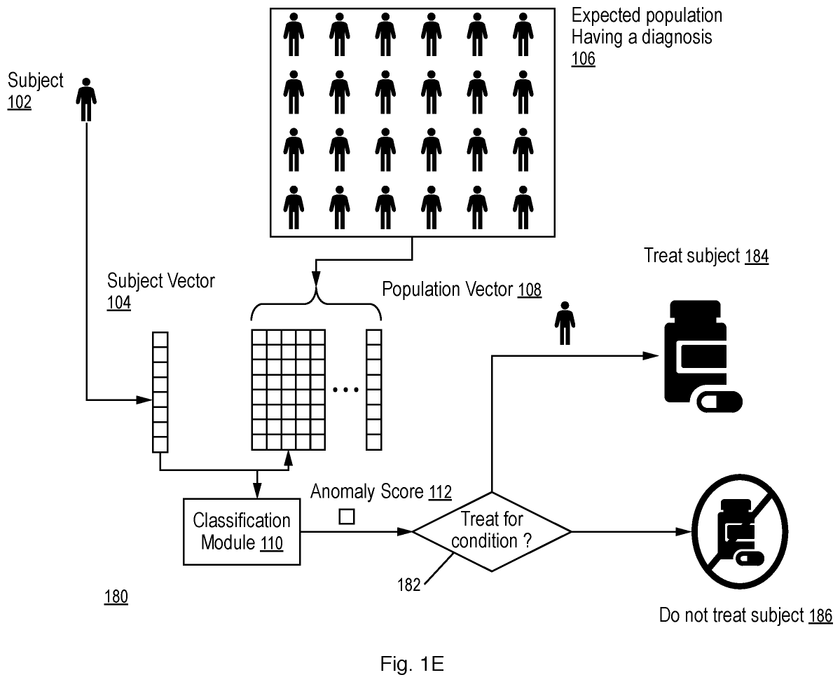

FIG. 1A is a block diagram 100 illustrating example embodiments of the present disclosure. In some embodiments, provided is a method that screens a subject 102 for eligibility for a study or treatment, or verifies a diagnosis of the subject 102. First, the subject 102 takes a diagnosis test having multiple elements, which generates a subject vector 104. As described above, the subject vector is a one-dimensional array of a plurality of values derived from the item scores in a rating scale for a single subject. As one example, the subject vector 104 can be individual elements from the subject taking the MADRS test.

In addition, an expected population having a diagnosis 106 has associated population vectors 108 having a vector of elements for each individual within the population 106. The population vectors 108 have the same elements as the subject vector 104 (e.g., the MADRS test), so that the subject vector 104 can be easily compared to the population vector 108. A classification module (e.g., a trained forest model) 110 compares the subject vector 104 to the population vector 108 by traversing decision trees and generates an anomaly score 112 based on the comparison, as described more in further detail herein. The anomaly score 112 is low when the subject vector 104 is close to the expected patterns as shown in the population vectors 108. However, the anomaly score is high when the subject vector 104 deviates from the population vectors 108. A person having ordinary skill in the art can recognize that, in some embodiments, the anomaly score 112 can be scaled differently, such that a high score allows the subject inclusion in the study/treatment and a low score excludes the subject from the study/treatment.

A pre-screening module 114 then screens the subject 102 based on the anomaly score 112. If the anomaly score is within a tolerable range (e.g., low enough), the subject can be included in the study 116. In some embodiments, provided is a method to verify the diagnosis, or approve treatment. However, if the anomaly score is outside of the tolerable range (e.g., too high), the subject is excluded from the study 118, diagnosis contradicted, or treatment denied.

FIG. 1B is a block diagram 120 illustrating example embodiments of the present disclosure. The method screens a subject 102 for improving a clinical dataset. First, the subject 102 takes a diagnosis test having multiple elements, which generates a subject vector 104. As described above, the subject vector is a one-dimensional array of a plurality of values derived from the item scores in a rating scale for a single subject. As one example, the subject vector 104 can be individual elements from the subject taking the MADRS test.

In addition, an expected population having a diagnosis 106 has associated population vectors 108 having a vector of elements for each individual within the population 106. The population vectors 108 have the same elements as the subject vector 104 (e.g., the MADRS test), so that the subject vector 104 can be easily compared to the population vector 108. A classification module (e.g., a trained forest model) 110 compares the subject vector 104 to the population vector 108 by traversing decision trees and generates an anomaly score 112 based on the comparison, as described more in further detail below. The anomaly score 112 is low when the subject vector 104 is close to the expected patterns as shown in the population vectors 108. However, the anomaly score is high when the subject vector 104 deviates from the population vectors 108. A person having ordinary skill in the art can recognize that, in some embodiments, the anomaly score 112 can be scaled differently, such that a high score allows the subject inclusion in the study/treatment and a low score excludes the subject from the study/treatment.

A population enrichment module 122 then screens the subject 102 based on the anomaly score 112. If the anomaly score is within a tolerable range (e.g., low enough), the subject, and the subject's associated data, can be included in the clinical dataset 124. However, if the anomaly score is outside of the tolerable range (e.g., too high), the subject and its associated data is excluded from the study 124.

FIG. 1C is a block diagram 140 illustrating example embodiments of the present disclosure. The method screens a subject 102 for verifying a diagnosis. In some embodiments, the subject 102 has been diagnosed with a disease or condition. In some embodiments, provided is a method to verify or reject the diagnosis of the disease or condition. First, the subject 102 takes a diagnosis test having multiple elements, which generates a subject vector 104. As described above, the subject vector is a one-dimensional array of a plurality of values derived from the item scores in a rating scale for a single subject. As one example, the subject vector 104 can be individual elements from the subject taking the MADRS test.

In addition, an expected population having a diagnosis 106 has associated population vectors 108 having a vector of elements for each individual within the population 106. The population vectors 108 have the same elements as the subject vector 104 (e.g., the MADRS test), so that the subject vector 104 can be easily compared to the population vector 108. A classification module (e.g., a trained forest model) 110 compares the subject vector 104 to the population vector 108 by traversing decision trees and generates an anomaly score 112 based on the comparison, as described more in further detail below. The anomaly score 112 is low when the subject vector 104 is close to the expected patterns as shown in the population vectors 108. However, the anomaly score is high when the subject vector 104 deviates from the population vectors 108. A person having ordinary skill in the art can recognize that, in some embodiments, the anomaly score 112 can be scaled differently, such that a high score allows the subject inclusion in the study/treatment and a low score excludes the subject from the study/treatment.

A diagnosis verification module 142 then screens the subject 102 based on the anomaly score 112. If the anomaly score is within a tolerable range (e.g., low enough), the diagnosis is confirmed 144. In response to the confirmed diagnosis, a clinician can treat the subject 102 for the disease or condition of the diagnosis, the subject 102 can be admitted to a clinical trial, or other action can result. However, if the anomaly score is outside of the tolerable range (e.g., too high), the diagnosis of the subject is rejected 146. In response to the rejected diagnosis, a clinician can seek a new diagnosis, the subject 102 be prevented from joining a clinical trial, or other action can result.

FIG. 1D is a block diagram 160 illustrating example embodiments of the present disclosure. The method screens a subject 102 to determine if a subject is an outlier (e.g., for eligibility for a study or treatment, or verifies a diagnosis of the subject 102). First, the subject 102 takes a diagnosis test having multiple elements, which generates a subject vector 104. As described above, the subject vector is a one-dimensional array of a plurality of values derived from the item scores in a rating scale for a single subject. As one example, the subject vector 104 can be individual elements from the subject taking the MADRS test.

In addition, an expected population having a diagnosis 106 has associated population vectors 108 having a vector of elements for each individual within the population 106. The population vectors 108 have the same elements as the subject vector 104 (e.g., the MADRS test), so that the subject vector 104 can be easily compared to the population vector 108. A classification module (e.g., a trained forest model) 110 compares the subject vector 104 to the population vector 108 by traversing decision trees and generates an anomaly score 112 based on the comparison, as described more in further detail below. The anomaly score 112 is low when the subject vector 104 is close to the expected patterns as shown in the population vectors 108. However, the anomaly score is high when the subject vector 104 deviates from the population vectors 108. A person having ordinary skill in the art can recognize that, in some embodiments, the anomaly score 112 can be scaled differently, such that a high score allows the subject inclusion in the study/treatment and a low score excludes the subject from the study/treatment.

A pre-screening module 162 then screens the subject 102 based on the anomaly score 112. If the anomaly score is within a tolerable range (e.g., low enough), the subject is not flagged as an outlier 164. The subject 102 can then be included in a clinical trial, receive treatment, or other action. However, if the anomaly score is outside of the tolerable range (e.g., too high), the subject 102 is identified as an outlier 166. In response to the subject 102 being identified as an outlier, the diagnosis of the subject 102 is contradicted, treatment for the diagnosis denied, inclusion in a clinical study denied, or other action can be performed.

FIG. 1E is a block diagram 180 illustrating example embodiments of the present disclosure. The method treats a subject 102 for a psychiatric condition. First, the subject 102 takes a diagnosis test having multiple elements, which generates a subject vector 104. As described above, the subject vector is a one-dimensional array of a plurality of values derived from the item scores in a rating scale for a single subject. As one example, the subject vector 104 can be individual elements from the subject taking the MADRS test.

In addition, an expected population having a diagnosis 106 has associated population vectors 108 having a vector of elements for each individual within the population 106. The population vectors 108 have the same elements as the subject vector 104 (e.g., the MADRS test), so that the subject vector 104 can be easily compared to the population vector 108. A classification module (e.g., a trained forest model) 110 compares the subject vector 104 to the population vector 108 by traversing decision trees and generates an anomaly score 112 based on the comparison, as described more in further detail below. The anomaly score 112 is low when the subject vector 104 is close to the expected patterns as shown in the population vectors 108. However, the anomaly score is high when the subject vector 104 deviates from the population vectors 108. A person having ordinary skill in the art can recognize that, in some embodiments, the anomaly score 112 can be scaled differently, such that a high score allows the subject inclusion in the study/treatment and a low score excludes the subject from the study/treatment.

A treatment screening module 182 then screens the subject 102 based on the anomaly score 112. If the anomaly score is within a tolerable range (e.g., low enough), the subject can be treated for the condition 184. Treatment can include administering a treatment such as a medicine or therapy, or being included in a clinical trial, however a person having ordinary skill in the art can recognize this list of treatments is nonexclusive and other treatments can be performed. However, if the anomaly score is outside of the tolerable range (e.g., too high), the subject 102 is denied treatment 186.

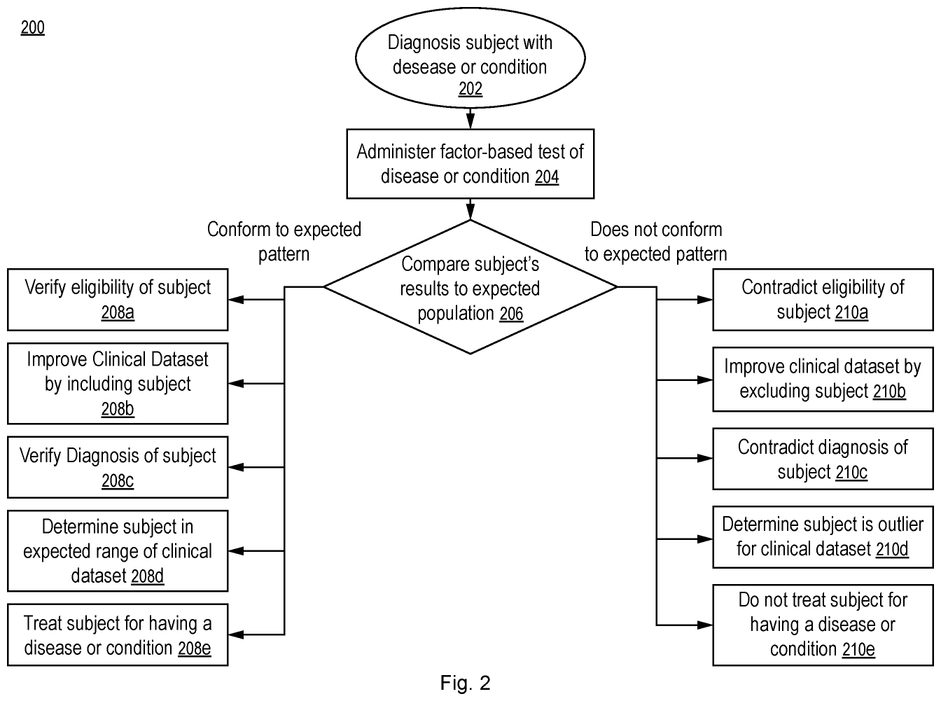

FIG. 2 is a flow diagram 200 illustrating example embodiments of methods employed by the present disclosure. A clinician diagnoses a subject with a disease or condition (e.g., of a psychiatric condition) (202). The clinician or another expert administers an element-based test of the disease or condition, such as MADRS or PANSS (204). In some embodiments, steps 204 can be performed before step 202. Then the method compares the results of the subject to that of an expected population (206). The comparison reveals either conformation with an expected pattern (208a-e) or deviation the expected pattern (210a-e).

When the comparison reveals conformation with the expected pattern, the method can verify eligibility of the subject (208a), improve a clinical dataset by including the subject (208b), verify a diagnosis of a subject (208c), determine subject is in expected range of clinical dataset (208d), or treat the subject for having the disease or condition (such as a psychiatric condition) (208e). A person having ordinary skill in the art can recognize that these elements are nonexclusive, and can be performed alone or in combination.

When the comparison reveals deviation from the expected pattern, the method can contradict eligibility of the subject (210a), improve the clinical dataset by excluding the subject (210b), contradict the diagnosis of the subject (210c), determine the subject is an outlier (210d), or deny treatment of the subject for having the disease or condition (such as a psychiatric condition) (210e). A person having ordinary skill in the art can recognize that these elements are nonexclusive, and can be performed alone or in combination.

In some embodiments, provided is a method employed as a means to determine an intent-to-treat (ITT) population. For example, a group of otherwise eligible subjects are screened in two parts. In a first part, the screening verifies the diagnosis by using evidence such as objective medical records or diagnosis by an expert (e.g., a psychiatrist), which rejects subjects having no support for the diagnosis. In a second part, the screening verifies the ongoing symptoms are consistent with canonical presentation of the condition using the multi-item test (e.g., MADRS, PANSS, etc.), in this instance, bipolar depression, and rejects subjects based on heterogeneous symptom presentation. Using both filters, a randomized intention-to-treat (ITT) sample of patients is created. It can be difficult to examine a subject's screening elements against baseline elements manually or by human eye alone, and therefore a rigorous and automated approach is needed. In some embodiments, provided is an outlier detection model that can be built by imputing symptom rating scores from each subject. The method computes item variances, pairwise covariances, angles, differences, or combinations as training set for the model.

In some embodiments, an automated approach can be a method that trains an isolation forest (i-forest) using bootstrap samples with optimal sizes (e.g., 128-256 per tree). The method builds multiple trees (e.g., 1,000-1,500 or more). Each tree has multiple branches. At each node, data is split based on random split vector to form more nodes. The method attempts to split data until each node contains one sample. Once all 1,000-1,500 trees are built, the method computes the average path length from the root node to an external node using all trees. The method then adjusts the average path length to reflect that the samples that are difficult to split have the longer path length. The method then inverts the adjusted path length to reach the anomaly score. In some embodiments, the anomaly inverted adjusted path length can be multiplied by a factor (e.g., 2) to reach the anomaly score). The method runs a simulation using different sub-sample size, and the number of trees to confirm the stability of the model. The method then fits the first final vector with the trained i-forest and computes an anomaly score. An i-forest is an unsupervised decision tree-based that can be used for to detect anomalies as described herein. In an embodiment, an anomaly threshold represents an optimal anomaly score that, when used as a cutoff value, maximizes the sample size of the population that conforms to the expected symptom patterns.

In some embodiments, the model outputs can be used in a predictive model for placebo response.

The forest can be trained on any of the following three datasets or combinations thereof for a first trial of a first treatment and second trial of a second treatment:
a) a first trial screening and baseline;
b) a first trial and second trial screening and baseline; and
c) a first trial and second trial post baseline.

After training, regardless of the dataset used for the training, the models are characterized by generating a lower anomaly when there is the more observed separation between the treatment and placebo.

In some embodiments, in contrast to a random forest, the split rule of an i-forest involves a random split value between the maximum and minimum values. The number of partitions required to isolate a data point x is equivalent to the path length, h(x), from the root node to an external node. The score is defined as:

$$score ( x , n ) = {2}^{\frac{- E ( h ( x ) )}{c ( n )}} where c ( n ) = 2 ( H ( n ) - 1 ) - 2 \frac{( n - 1 )}{n} ,$$

which is the estimated average of h(x) given external n nodes, where H is the harmonic number of n. The adjustment of h(x) is to account for the fact that the average height of the estimator grows in the order of log n. In some embodiments, the threshold of anomaly scores is determined based on goodness-of-fit of the training data to established factor models and expected proportion of anomaly subjects in the population.

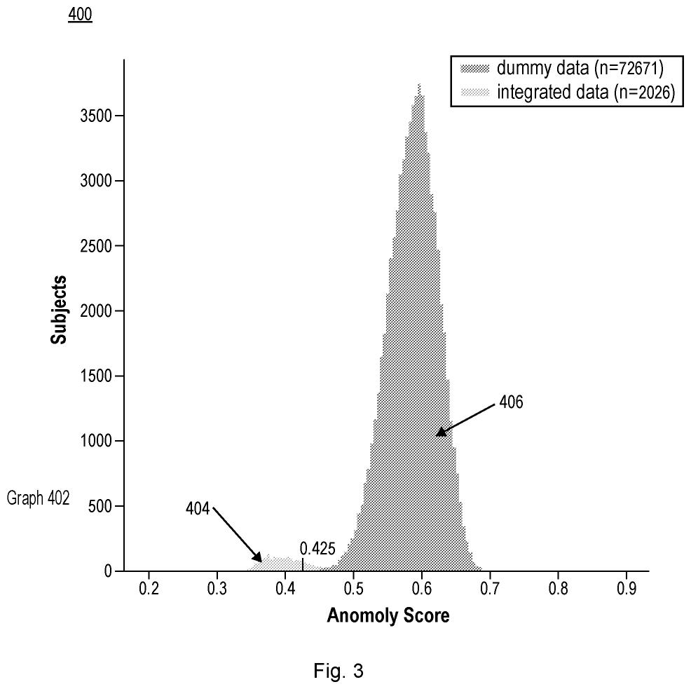

FIG. 3 is a diagram 400 illustrating anomaly scores separated from decoy subject's ratings from actual subject's ratings. In graph 402, decoy subjects having randomized MADRS scores are shown to have a distribution of extremely high anomaly scores, where study subjects are shown to have low anomaly scores.

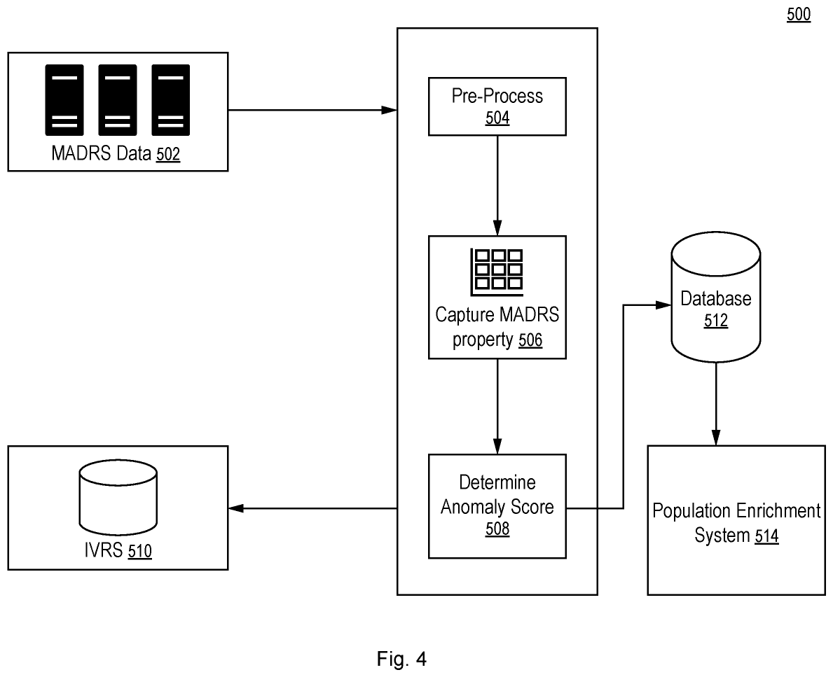

FIG. 4 is a block diagram 500 illustrating an example embodiment of the present disclosure. MADRS Data 502, stored in one or more databases, and can be processed locally or by one or more cloud server. The method pre-processes 504 the MADRS data 502 (e.g., calculating vectors as described above) and enables capturing MADRS properties. The method then determines an anomaly score 508 for each subject, which is transferred to a database 512. A population enrichment system 514 can then apply the anomaly scores, as described above. One example application is applying the anomaly scores to a clinical study. In some embodiments, the anomaly scores can also be used by interactive voice response systems (IVRS) or interactive web response systems (IWRS) 510 that administer clinical trials. In some embodiments, the population enrichment system can interact with the IVRS 510 to provide the anomaly scores.

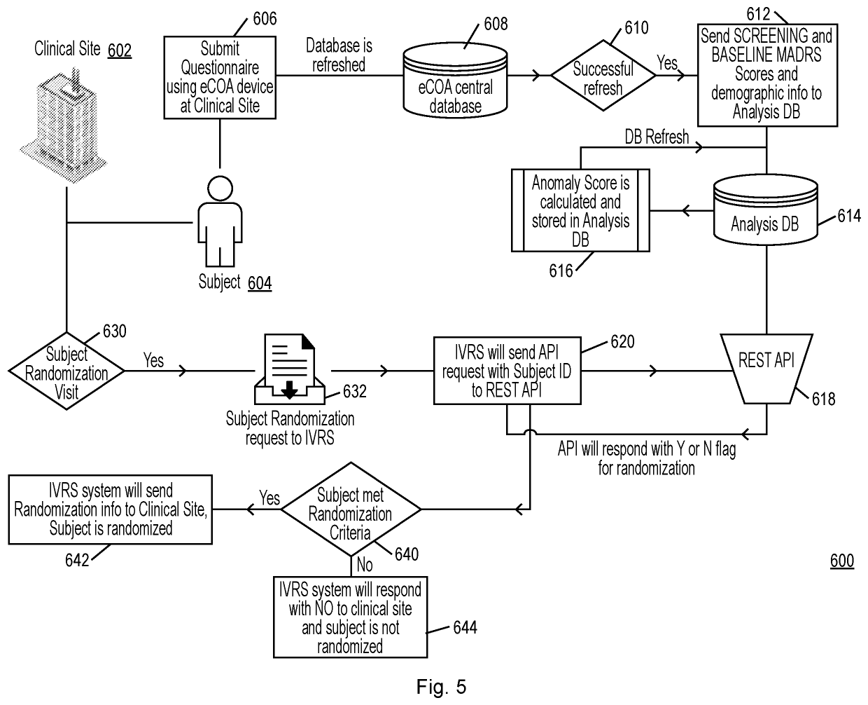

FIG. 5 is a block diagram 600 illustrating an example embodiment of the present disclosure. In some embodiments, a method is provided for a subject 604 submitting a questionnaire at a clinical site 602 (606). In some embodiments, the subject 604 can submit the questionnaire using an electronic clinical outcome assessment (eCOA) device (606). The results of the study can be sent to a eCOA central database (608). After a successful refresh of the database (610), the method sends screening and baseline MADRS scores and demographic information of the subject 604 to an analysis central database (612). Once stored in the central database, an anomaly score can be calculated and stored in the analysis central database 614 (616).

Meanwhile, in response to the subject's 604 visit to the clinical site 602, the clinical site creates a subject randomization visit record (630). In response to creation of the record, the method submits a randomization request to the IVRS system (632). An Application Programming Interface (API) is configured to produce flags allowing or excluding a subject based on the anomaly scores in the analysis central database 614 (618). The API 618 sends the flags in response to an IVRS sending an API request with the subject ID to the API (620).

If the subject meets the randomization criteria (640), the IVRS system sends randomization information to the clinical site 602 and the subject is randomized (642). If the subject does not meet the randomization criteria (640), the IVRS system responds with “NO” to the clinical site and the subject is not randomized (644).

Drug trials for negative symptoms in schizophrenia select for patients based on severity and stability of negative symptoms relative to positive symptoms using criteria which are not suitable for trials of acute exacerbation of schizophrenia. In some embodiments, presented herein is a general method to enrich for subjects having a specific predefined factor structure in PANSS, which is applicable to any trial population. In some embodiment, a vector of 1335 elements based on between- and within-item variance, covariance and differences of PANSS items used to calculate an index of heterogeneity for each of any predetermined symptom construct in PANSS. Using pre-randomization data, enrichment can be demonstrated for subjects who maintain the maximal variance explained by the 7-item of the Marder PANSS negative symptom (MPNS) construct that is robust and generalizable across N=4,876 subjects in 13 trials of acute schizophrenia. In a retrospective application to an acute trial of ulotaront, a TAAR1 agonist, the improvements in negative symptoms seen in the total population appeared to be specific to the approximately 20% of subjects identified prior to randomization as having the maximum MPNS construct. In contrast, in 5 acute trials of lurasidone, a dopamine D2-based antipsychotic, the improvements on negative symptoms appeared to lack specificity for this subpopulation. These results demonstrate that psychometric properties of PANSS derived from factor analyses in large sample sizes, can be used at the individual subject level and applied towards prognostic enrichment of clinical trials.

The development of drugs for negative symptoms has been hampered by several methodologic challenges. One challenge is differentiating primary negative symptoms (attributable to the underlying neurobiology of schizophrenia) from secondary negative symptoms. Negative symptoms are frequently secondary to (1) the acute effects of positive symptoms, (2) the presence of depression or anxiety, and/or (3) the adverse effect of D2 antagonist drugs. Each of these three factors may result in negative symptoms (e.g., blunted affect, alogia, apathy, avolition, asociality) that are both difficult to distinguish from primary negative symptoms, and whose improvement may result in the incorrect inference that a drug has specific efficacy in treating negative symptoms (an inference that the FDA has characterized as being due to “pseudospecificity”). Also, within a population of acutely psychotic patients only a proportion, perhaps 50-60%, have prominent negative symptoms. In an attempt to identify an appropriate target population with primary negative symptoms, the few clinical trials that have been conducted have generally restricted study entry to stable outpatients with persistent negative symptoms who have low levels of positive symptoms. These trials have been difficult to implement. Limiting negative symptom trials to stable patients with low-grade symptomatology and persistent negative symptoms may be useful for differentiating primary versus secondary negative symptoms, however excluding acutely psychotic patients from treatment studies of negative symptoms is likely to exclude a clinically important population, and brings no clear benefit in measurement of adjusted negative symptom change, nor reduces the correlations between negative and positive symptom change. Currently, studies of negative symptoms in acutely psychotic patients have employed a post-hoc analysis strategy that examined efficacy in the subgroup of patients with “predominant” negative symptoms, where the severity of negative symptoms is greater than the severity of positive symptoms.

Valid measurement of negative symptoms also represents an important methodologic challenge. Over the past 15 years, several scales measuring negative symptoms have been validated, for example, the Brief Negative Symptom Scale (BNSS), which has demonstrated good levels of discriminant validity versus the PANSS Positive subscale score (r=0.09). Nonetheless, the newer negative symptom scales have not been widely used in registration trials. Furthermore, they were not designed to discriminate between primary and secondary negative symptoms.

Since the Positive and Negative Syndrome Scale (PANSS) continues to be, by far, the most widely used primary outcome measure in clinical trials, we have developed a factor analytic approach that applied weighted coefficients to each PANSS item to generate PANSS factors (UPSM) that exhibit low levels of between-factor correlation (with r-values in the range of 0.04-0.10 for UPSM negative symptoms versus both the positive and depression/anxiety factors), and yet have high face validity and show minimal loss of information when compared to the original Marder PANSS factors. Drug vs. placebo effect sizes based on UPSM PANSS factors are specific for individual symptom domains because each UPSM factor has minimal correlation with other factors UPSM PANSS factors.

Schizophrenia is a heterogeneous psychiatric disorder characterized by multiple distinct symptom domains. The positive symptoms of delusions, hallucinations, and disorganized speech, contrast with the negative symptoms of diminished emotional expression and apathy/avolition. To date, however, it has been difficult to describe the differences in the clinical presentation of this disorder as a function of its biological heterogeneity. Attempts to define more-homogeneous clinical subtypes have lacked diagnostic and prognostic utility In the absence of clinically-useful understanding of heterogeneity, diagnosis and prognosis at the level of individual subjects mainly focus on functional impairment associated with symptom severity (e.g., level of functioning in work, interpersonal relations, and self-care) and on symptom stability (e.g., duration of symptoms over time), rather than on symptom heterogeneity (e.g., the relationships within a subject between contrasting symptom domains).

Without reliable biological markers, research studies continue to rely on rating scales designed to measure the severity of total symptoms across symptom domains. Severity and stability are often assessed in research studies with the widely-adopted rating scale Positive and Negative Syndrome Scale (PANSS). The 30 items of the PANSS assess severity across multiple symptom dimensions, with a higher total score indicating higher overall severity across domains. Factor analyses of PANSS reliably identify the dimensions of positive, negative, disorganized, hostile, and mood symptoms. Clinical studies of negative symptoms in patients with schizophrenia traditionally use items from the PANSS to define entry criteria based on the relative severity of negative versus positive symptoms. However, since the currently-accepted criteria for Predominant/Prominent Negative Symptoms are based on severities of PANSS items, changes in total scores confound specific effects on items within PANSS. Currently, recommendations for clinical trials evaluating treatment effects on negative symptoms are forced to include periods to demonstrate stability in total symptoms.

Efforts to improve the psychometric properties of scales to measure negative symptoms continue, but meanwhile, novel analysis strategies are needed with existing scales to understand specificity, stability, and subtypes. An Uncorrelated PANSS Score Matrix (UPSM) is disclosed that can dissect overall drug-placebo treatment effect sizes on PANSS total score into specific components at the level of specific symptom domains, independent from correlated changes expected from the effect on total symptoms. The method of UPSM was also able to dissect specificity of drug-placebo treatment effect in sub-populations of patients expressing symptom predominance within unique domains. These methods of analysis rely on relatively large sample sizes available in drug registration trials, and have not been adapted at the level of the individual subject to enrich clinical trials for targeted symptom domains (e.g., negative or positive symptoms). Current methods lack symptom-based, data-analytical approaches to relate properties of individual subjects at study entry to the known heterogeneity of the disorder. The development of such enrichment strategies to target specific study populations can facilitate the development and characterization of novel treatments in schizophrenia.

Heterogeneity at the population level is useful for research studies. Large clinical samples can exploit heterogeneity to reveal the dimensions of underlying psychopathologies. For example, by analyzing the variance explained between subjects in large samples, factor analyses of rating scales can reveal the dimensions of positive, negative, and cognitive symptoms. Correlations of item severities within a population demonstrate clustering and their shared variance are thought to reflect a shared psychopathology. Such relationships can be determined at the population level independent from the severity of total symptoms per se. However, at the level of individual subjects, heterogeneity hinders the understanding of individual psychopathology because item severities within- and between-symptom domains are so highly dependent on total scores. However, if symptom heterogeneity are characterized at the level of individual subjects using established rating scales, then the resulting increased understanding of schizophrenia symptom measurement can facilitate the development of novel treatments. In an embodiment, methods are disclosed herein that are applicable at the individual subject level for understanding psychopathology of specific symptom domains independent of symptom severity itself.

Treatment of schizophrenia has largely focused on reducing positive symptoms, however a substantial portion of the psychopathology is accounted for by negative symptoms. Consider a hypothetical subpopulation of patients who may have substantially more symptom variance explained by the 7 Marder negative PANSS items, than is observed for a population taken as a whole. To identify such patients, a method can employ a mathematical, vector-based approach to analyze PANSS items from individual subjects to quantify their heterogeneity. The method utilizes within-subject PANSS data between two sequential assessment periods prior to randomization (screening and baseline). The ability to prognostically enrich for a specific dimension of psychopathology, independent of total item scores and, at the level of individual subjects is a powerful strategy for uncovering specific drug-treatment effects in clinical trials. Using clinical trial data evidence for specific treatment effects on certain symptom domains (e.g., negative symptom domain, anxiety symptom domains) may be demonstrated in trials of patients with an acute exacerbation of schizophrenia.

Subject-level PANSS item scores between two assessments (screening and baseline) were encoded in a variance-covariance difference (VCD) vector. The VCD vector captures the intra-item variance, between-item covariance, and between-item differences of PANSS items between two assessment time points from a single subject. Briefly, for each subject h, a variance-covariance matrix of 30 PANSS items as consisting of the unique elements was defined as

$$V = [ \begin{matrix}{σ}_{{s}_{1}}^{2} & … & {σ}_{{s}_{1 , 30}} \\ ⋮ & ⋱ & ⋮ \\ {σ}_{{s}_{30 , 1}} & … & {σ}_{{s}_{30}}^{2}\end{matrix} ]$$

$$where$$

$${σ}_{{s}_{j}}^{2} = {\sum }_{t = 1}^{2} {( {s}_{t}^{j} - \overline{{s}^{j}} )}^{2}$$

is the un-biased variance of s¹, j=1, 2, . . . , 30, and

σₛ_{_{i,j}}=Σₜ₌₁²(sₜⁱ−s^{l̅})(sₜ^{j}−s^{j̅}),
is the unbiased covariance of PANSS items i and j, and

$$\overline{{s}^{j}} = \frac{{\sum }_{t = 1}^{2} {s}_{t}}{2} .$$

Note that the denominator is 2−1=1 for two time points. The unique elements of V for subject h were kept in vector u_{cov}_{ₕ}, consisting of elements of V on and below the main diagonal.

Separately, a difference matrix for 30 PANSS items was defined as

$$D = [ \begin{matrix}{d}_{{s}_{1 , 1}} & … & {d}_{{s}_{1 , 30}} \\ ⋮ & ⋱ & ⋮ \\ {d}_{{s}_{30 , 1}} & … & {d}_{{s}_{30 , 30}}^{2}\end{matrix} ]$$

where dₛ_{_{i,j}}=sⁱ−s^{j} for scores of items i and j. Note that the diagonal elements of D are 0. The unique elements of D for subject h at timepoint t are kept in vector dₜ(h), consisting of elements of D below the main diagonal.

Together, the VCD vector of Subject h for 2 timepoints (e.g. screening and baseline) is therefore defined as

VCDVₕ_{_{(t=1,t=1)}}=[u_{cov}_{ₕ}d₁₍ₕ₎,d₂₍ₕ₎]→ [symbol] ^{1,1335}
and VCD vector for N subjects is

VCDV_{(t=1,t=2)}=[VCDVₕ_{_{1(t=1,t=2)}}VCDVₕ_{_{2(t=1,t=2)}} . . . VCDVₕ_{_{N(t=1,t=2)}}]→ [symbol] ^{N,1335}

Using the PANSS-defined VCD vector, and the 7 items of the Marder PANSS negative symptoms factor, a new vector of 84 elements per subject was used to define a Marder negative heterogeneity index (MNHI). Table 1 lists the parameters used to derive the Marder Negative Heterogeneity index.

TABLE 1
Parameters to derive the Marder Negative Heterogeneity Index (MNHI).

| | |
|---|---|
| σₛ_{ᵢ}² | Variance of PANSS item i between screening and |
| | baseline of subject h |
| covᵢ_{ₛ}_{,i}_{_{b}} | Covariance of PANSS item i between screening |
| | and baseline of subject h |
| C(x) | Count of x |
| dₛ_{_{i,j}} | Difference between PANSS item i and PANSS |
| | item j of subject h |
| p | Set of combinations of two Marder negative items |
| $Δ {σ}_{{s}_{{( i , j )}_{v}}}^{2}$ | σₛ_{ᵢ}² − σₛ_{ₖⱼ}² of subject h at visit = t |

Marder's seven negative symptoms are congruent based on Marder factor model. Therefore, σₛ_{ᵢ}²−σₛ_{ⱼ}² is expected to be small for all p combinations. Similarly,

$$Δ {σ}_{{s}_{{( i , j )}_{v}}}^{2}$$

is expected to be smaller for all p combinations at v=screening and v=baseline. Furthermore, C(σₛ_{_{i,j}}|<0) is expected to be smaller for all Marder negative symptoms. Hence, the raw Marder Negative Heterogeneity Index (rMNHI) of subject h was defined as the sum of L1 norm of variance differences, count of negative covariance, L1 norm of between item differences at screening and baseline. It can be expressed as

$${rMNHI}_{h} = {‖ {Δσ}_{{s}_{P}}^{2} ‖}_{1} + {\sum }_{p = 1}^{21} C ( {cov}_{p} \vert < 0 ) + {‖ {d}_{{s}_{p , t = 1}} ‖}_{1} + {‖ {d}_{{s}_{p , t = 2}} ‖}_{1}$$

then min-max scaling (min=0, max=223) was applied to rMNHI to derive MNHI of subject h.

In principle, the methods may be applied to enrich for any other item-level construct as a universal approach to any psychometric assessment scale.

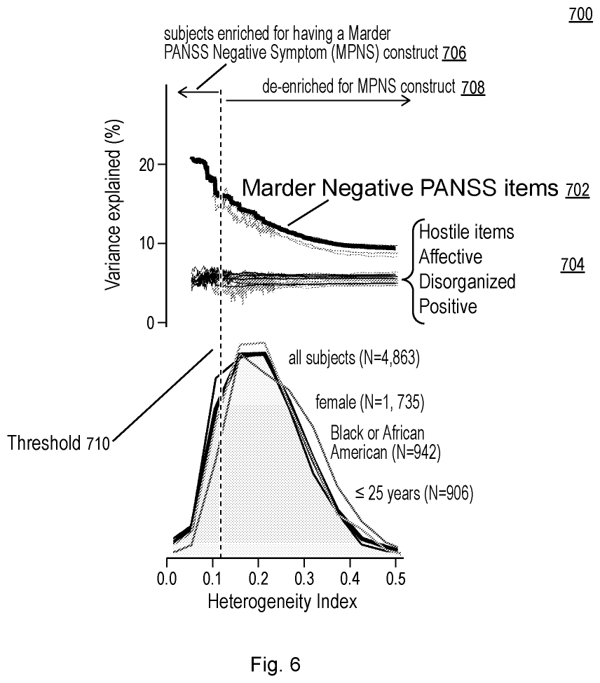

FIG. 6 is a graph 700 illustrating a threshold for enriching subjects for having a Marder PANSS Negative Symptom (MPNS) construct. A threshold 710 separates subjects enriched for having a MPNS construct 706 and subjects de-enriched for having an MPNS construct 708. In this example, the threshold is slightly above heterogeneity index 0.1, but a person having ordinary skill in the art can recognize that other thresholds can be employed.

The graph illustrates the percentage of variance explained for Marder PANSS items 702 and corresponding moods 704, the moods being hostile items, affective, disorganized, and positive.

PANSS assessments prior to randomization (screening, baseline) were pooled for ITT populations of 13 studies in schizophrenia (Table 2).

TABLE 2
List of studies in acute schizophrenia

| NCT | N | Weeks | Treatment | Geographies | Age Group |
|---|---|---|---|---|---|
| | 146 | 6 | treatment 1 | USA | |
| NCT00044044 | 349 | 6 | treatment 1, | USA | |
| | | | treatment 2 | | |
| NCT00711269 | 455 | 6 | treatment 1, | Japan/Korea/Taiwan | |
| | | | treatment 3 | | |
| NCT00088634 | 180 | 6 | treatment 1 | USA | |
| NCT00549718 | 489 | 6 | treatment 1 | USA/ROW | |
| NCT00615433 | 473 | 6 | treatment 1, | USA/ROW | |
| | | | treatment 4 | | |
| NCT00790192 | 482 | 6 | treatment 1, | USA/ROW | |
| | | | treatment 5 | | |
| NCT01911429 | 326 | 6 | treatment 1 | USA/ROW | adolescents 13-17 |
| NCT01821378 | 411 | 6 | treatment 1 | USA/ROW | |
| NCT01614899 | 455 | 6 | treatment 1 | Japan/Korea/Taiwan/ | |
| | | | | Malaysia | |
| | 478 | 6 | treatment 1 | Japan/Poland/Romania/ | |
| | | | | Russia/Ukraine | |
| NCT02002832 | 384 | 6 | treatment 1 | China | |
| NCT02969382 | 245 | 4 | treatment 6 | USA/ROW | adults <40 |
| | 4,868 | | | | |

Marder's one-factor model was used for the confirmatory factor analysis (CFA) to investigate the subjects contributing to good fit. Confirmatory factor analysis (CFA) was performed using a R package lavaan, using maximum likelihood estimation (MLE) with robust Huber-White standard errors and a scaled test statistic that is asymptotically equal to the Yuan-Bentler T2-star test statistics. The estimation was selected to reduce the deleterious effects of multivariate non-normality. The Wishart likelihood approach was used in which the covariance matrix is divided by N−1, and both standard errors and test statistics are based on N−1. Goodness of fit indices, comparative fit index (CFI>0.95 indicating good fit), root mean square error of approximation (RMSEA<0.08), and Tucker-Lewis index (TLI>0.95) were computed.

Permutation testing was performed 100,000 times on the difference between the proportion of predominant negative subjects in enriched and non-enriched. The null hypothesis is that the difference of the proportion of predominant negative subjects in the enriched and non-enriched sample remains similar over treatment duration.

Treatment effect sizes were calculated for the UPSM-transformed PANSS scores and for PANSS total scores using PROC MIXED procedure in SAS 9.4 adjusted for baseline and country. Drug-placebo treatment effect sizes were calculated as the LS mean difference divided by the pooled standard deviation, obtained as the standard error of the LS mean difference divided by the square root of the sum of inverse treatment group sample sizes.

### Results

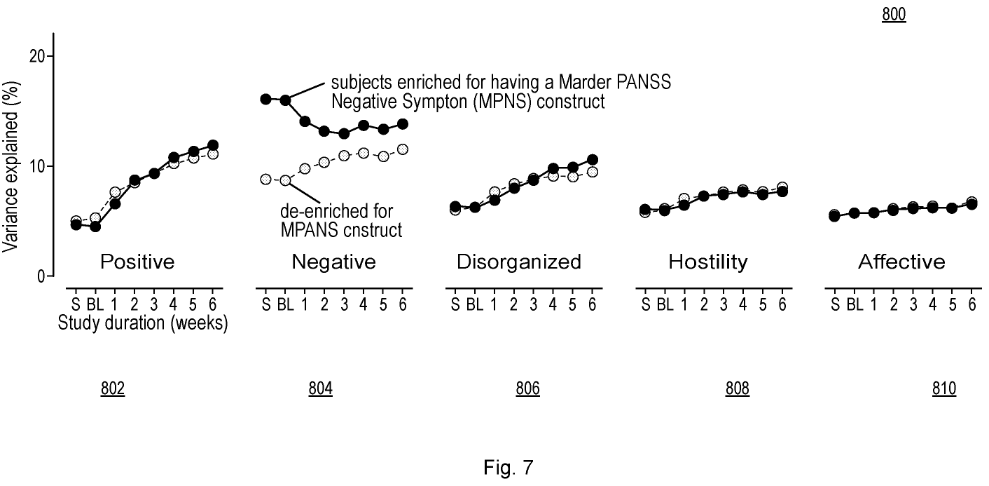

FIG. 7 are graphs 800 illustrating variances explained based on various factors on the PANSS scale. Each point on graph 802 represents subjects enriched for having a Marder PANSS Positive Symptom (MPPS) construct. Each point on graph 804 represents subjects enriched for having a Marder PANSS Negative Symptom (MPNS) construct. Each point on graph 806 represents subjects enriched for having a Marder PANSS Disorganized Symptom (MPDS) construct. Each point on graph 808 represents subjects enriched for having a Marder PANSS Hostility Symptom (MPHS) construct. Each point on graph 810 represents subjects enriched for having a Marder PANSS Affective Symptom (MPAS) construct.

Sufficient information on the factor structure in PANSS was expected to be contained within the PANSS assessments from a single subject between two time points, such that subpopulations enriched for a specified PANSS factor can be identified and enrolled one subject at a time. Factor analyses of PANSS data in trials of acute schizophrenia typically identify seven items of PANSS as the Marder Negative Symptom Factor. In trials of acute schizophrenia at baseline, the amount of variance explained by the Marder negative factor in a 5-factor model of PANSS is typically 10-20%. In the present disclosure and illustrated by FIG. 7, an a priori method identifies subjects, one at a time, prior to randomization, that when taken as a subpopulation, are presenting with a maximal amount of variance explained by the Marder Negative Symptom Factor.

FIG. 7 illustrates individual subjects (N=4,863) in acute schizophrenia trials were sorted by an index of heterogeneity calculated from each subject's PANSS-defined variance, covariance and difference vector, prior to randomization at the screening and baseline PANSS assessments. Confirmatory factor analysis (CFA) on each equally populated bins (N=253 subjects per bin) demonstrated that the amount of variance explained by the Marder Negative Symptom Factor was maximal (20%) for a subset (20%) of subjects in acute schizophrenia trials. The variance explained by the other PANSS factors remained constant as a function of the negative symptom heterogeneity index. FIG. 7 illustrates the ability to sort subjects based on individual heterogeneity on a single PANSS factor remained similar among demographic subgroups.

High factor loadings (above ˜0.5) and low unique variances (below 0.3) were evident in the enriched subpopulation (Table 3) with excellent indices of fit (CFI 0.99, TLI 0.98, and RMSEA of 0.07) indicative of a congruent Marder negative symptom factor structure (Table 3). The N=4,863 subjects have 40% variance explained by the 7 Marder negative items in a one-factor model, whereas the enriched subpopulation this amount of variance increases up to 69% and the remaining “de-enriched” subjects had only 38% variance explained by the negative symptoms factor model. The enriched subpopulation is defined as having a Marder PANSS Negative Structure (MPNS) construct.

Current definitions of negative symptoms rely on relative severity of negative items versus positive items, and thus classifications of subjects can change as total symptoms change (decrease) during the acute treatment phase. Using these methods, it can be examined whether the approach of defining negative symptoms as variance explained (MPNS subjects), rather than as total symptoms (e.g., Predominant Negative Symptoms), would provide a more-stable classification than current definitions. FIG. 7 illustrates that the enriched subjects remain distinct post randomization.

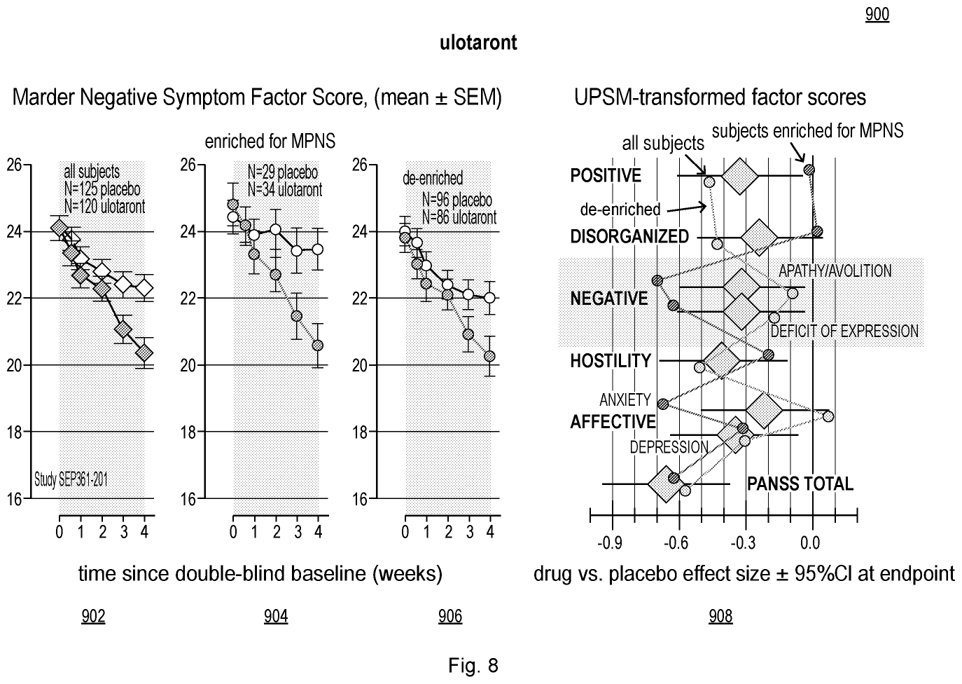

FIG. 8 are graphs 900 illustrating factor scores for a study of the drug ulotaront. Graph 902 illustrates the Marder Negative PANSS Symptom Factor Score (MPNS) for all subjects as measured at each week during a double-blind baseline study for 125 placebo patients and 120 patients being administered ulotaront. Graph 904 illustrates the Marder Negative PANSS Symptom Factor Score (MPNS) for subjects enriched for MPNS as measured at each week during a double-blind baseline study for 29 placebo patients and 34 patients being administered ulotaront. Graph 906 illustrates the Marder Negative PANSS Symptom Factor Score (MPNS) for subjects de-enriched for MPNS as measured at each week during a double-blind baseline study for 96 placebo patients and 86 patients being administered ulotaront.

Graph 908 is a graph illustrating UPSM-transformed factor scores. UPSM transformed factor scores illustrate drug v. placebo effect size to a 95% confidence interval at each endpoint. The graph illustrates the UPSM-transformed factor scores for positive, disorganized, negative, hostility, affective, and PANSS total scores. The graph illustrates the results for all subjects (in the diamond), enriched subjects, and de-enriched subjects.

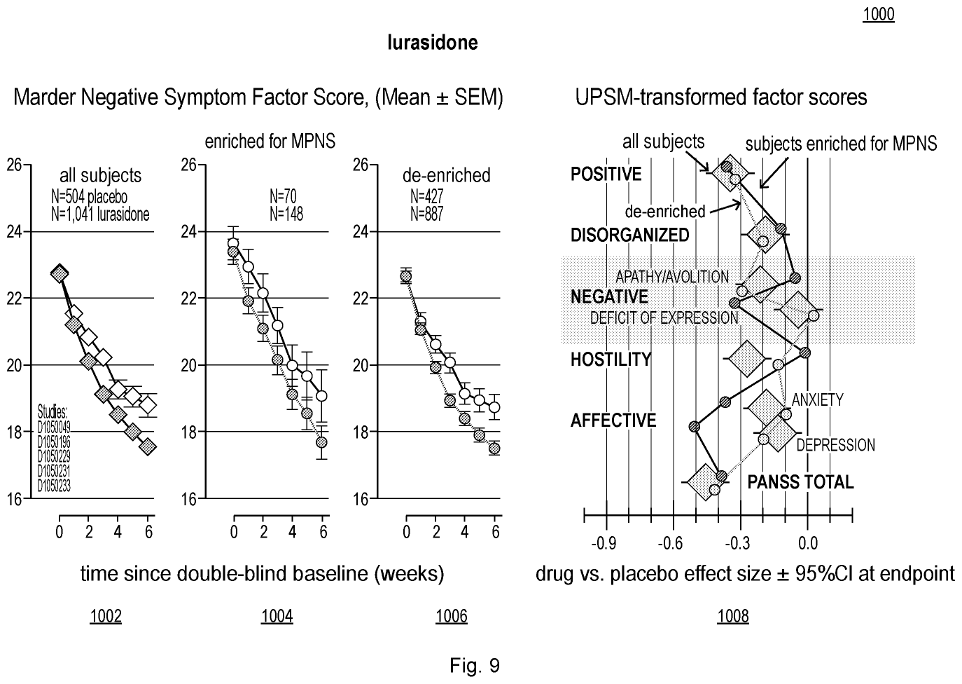

FIG. 9 are graphs 1000 illustrating factor scores for a study of the drug lurasidone. Graph 1002 illustrates the Marder Negative PANSS Symptom Factor Score (MPNS) for all subjects as measured at each week during a double-blind baseline study for 504 placebo patients and 1041 patients being administered lurasidone. Graph 1004 illustrates the Marder Negative PANSS Symptom Factor Score (MPNS) for subjects enriched for MPNS as measured at each week during a double-blind baseline study for 70 placebo patients and 148 patients being administered ulotaront. Graph 1006 illustrates the Marder Negative PANSS Symptom Factor Score (MPNS) for subjects de-enriched for MPNS as measured at each week during a double-blind baseline study for 427 placebo patients and 887 patients being administered ulotaront.

Graph 1008 is a graph illustrating UPSM-transformed factor scores. UPSM transformed factor scores illustrate drug v. placebo effect size to a 95% confidence interval at each endpoint. The graph illustrates the UPSM-transformed factor scores for positive, disorganized, negative, hostility, affective, and PANSS total scores. The graph illustrates the results for all subjects (in the diamond), enriched subjects, and de-enriched subjects.

Negative symptoms are a target of treatment especially for development-stage compounds having mechanisms of action that do not block dopamine D2 receptors. However, overall improvements in total symptoms obscure the specificity of improvements in negative symptoms, due to correlated improvements among items in PANSS. It was hypothesized that specific effects on negative symptoms may be more accurately determined in a subpopulation enriched for having the MPNS construct versus the larger population de-enriched for the MPNS construct, depending on the pharmacological mode of action. In subjects having the MPNS construct, versus those without, there was a similar benefit of drug treatment on total symptoms. For example, FIGS. 8-9 illustrate that PANSS total score effect sizes were similar. FIGS. 8-9 also illustrate that, in all subjects, drug-placebo separation was evident on the Negative Symptom Factor Score, as expected from an overall improvement in PANSS total score (PANSS Negative Symptom factor scores).

Experiments then tested for the specificity of the treatment effect on each of the domains of schizophrenia, using an Uncorrelated PANSS Score Matrix (UPSM) to transform PANSS. Ulotaront, a non-D2 compound, demonstrated specific treatment effects on negative symptoms, using the UPSM transformation of PANSS. FIG. 8 illustrates that in the current analysis, the same UPSM transformation was tested, but for the subjects enriched for having the MPNS construct prior to randomization.

### Discussion

When starting with an acutely psychotic patient population, it is difficult to attribute symptom change in schizophrenia to improvements in specific symptom domains (e.g., negative symptoms). The methods disclosed herein demonstrate that individual subjects can be selected prior to randomization to enrich for study populations with the greatest degree of variance explained by their negative symptom measurement, and thus facilitate the demonstration of specific treatment effects on negative symptoms. In principle, the approach can enrich for any predetermined symptom construct to address heterogeneity in clinical trials.

The structure of schizophrenia symptoms at baseline can be related to the structure of symptom change apparent over time postbaseline. Information on the structure of schizophrenia symptoms can also be contained in the item scores of individual subjects, assessed at two time points prior to randomization (screening and baseline). Using such an a priori rationale, and the desire to quantify heterogeneity along a single symptom domain, this disclosure develops a mathematical index to quantify heterogeneity isolated to a single dimension in PANSS. The heterogeneity detector was designed to be applied before randomization, based only on symptom presentation, and one subject at a time. The heterogeneity detector was envisioned to meet 3 criteria: (1) capture factor-analytical properties PANSS at the subject-level, rather than needing a population at larger sample size for those properties to emerge like factor analysis requires; (2) rank-order subjects by heterogeneity along a single PANSS factor (e.g., negative symptoms); (3) enrich for subjects who have a large variance explained by the pre-specified PANSS factor (e.g., the 7 items of the Marder PANSS negative factor structure).

The PANSS heterogeneity detector enriches for specific subpopulations of schizophrenia expressing variance along a specified symptom dimension. Symptom dimensions in PANSS data are more-typically revealed by factor analyses in large samples of PANSS data using the variance-covariance matrix of PANSS item scores. The PANSS heterogeneity detector also relies on a variance-covariance concept, but defined on individual subjects. For each subject's individual variance-covariance-difference vector, the 7 Marder negative items in PANSS are mathematically combined into a single index. The index of negative symptom heterogeneity is robust enough to rank-order subjects based on the amount of variance explained by their negative symptoms. The threshold value of Marder negative symptom heterogeneity index (0.119) identified here was selected based on maximizing the amount of variance explained on the MPNS construct in a selected subpopulation. The ability to choose patients whose variance in a specified symptom domain (negative symptoms) is well-described by the selected instrument (e.g., PANSS Negative Symptom Factor Score), improves the psychometric reliability of a selected endpoint in schizophrenia clinical trials.

Clinical trials designed to evaluate negative symptoms of schizophrenia seek to define stability with respect to the construct being measured. In this disclosure, a method includes a prognostic enrichment strategy for clinical trials of negative symptoms in schizophrenia, targeting a population more likely to have a pre-defined psychopathological construct. The method enriches for subjects having a construct defined by the 7-items of the Marder PANSS negative factor, where a specific drug effect on negative symptoms might be more readily demonstrated. In contrast, a prognostic enrichment strategy is proposed herein for clinical trials of negative symptoms in schizophrenia with a population more likely to have the psychopathological construct defined by the 7-items of the Marder PANSS negative factor, in which a specific drug effect on negative symptoms might be more readily demonstrated. Such a subpopulation would have minimal variance from other symptom domains contributing to the measurement of the targeted negative items, even post randomization, as demonstrated here where the subjects identified as having the MPNS construct at baseline also had greater variance explained by their negative symptoms post-randomization, as the acute treatment phase subsided, and as total symptoms decreased.

In clinical trials and in clinical practice, improvements in positive symptoms of schizophrenia likely contribute to improvements in negative symptoms. In drug registration trials of patients with an acute exacerbation of schizophrenia, for example, improvements in PANSS positive and negative subscale (factor) scores are highly correlated in their change from baseline to a 6-week endpoint. Thus, any efforts to improve psychometric instruments will have change scores correlated to changes in other symptom domains. One approach to addressing correlated changes among symptom domains, is to use an Uncorrelated PANSS Score Matrix (UPSM) to transform the item scores of PANSS and describe drug-placebo differences that are independent of correlated change in other domains.

The analytical approaches piloted here with already-conducted clinical trials, can be prospectively defined as analyses in clinical trials of acute exacerbation of schizophrenia, to facilitate the characterization of compounds with non-D2 mechanisms of action.

TABLE 3
Factor loadings on subjects enriched for Marder
PANSS Negative Symptom (MPNS) construct

| | | | De- | | |
|---|---|---|---|---|---|
| | | Enriched | enriched | | Not |
| ITT | | for | (not | Predominant | Predominant |
| population | | MPNS | MPNS) | Negative | Negative |
| N = 4,863 | Items of MPNS | N = 929 | N = 3,934 | N = 3,401 | N = 1,462 |
| 0.77 | N02 Emotional | 0.88 | 0.75 | 0.71 | 0.46 |
| | withdrawal | | | | |
| 0.71 | N04 | 0.88 | 0.69 | 0.63 | 0.43 |
| | Passive/Apathetic | | | | |
| | social avoidance | | | | |
| 0.69 | N01 Blunted affect | 0.85 | 0.67 | 0.58 | 0.53 |
| 0.67 | N06 Lack of | 0.84 | 0.65 | 0.56 | 0.56 |
| | spontaneity and | | | | |
| | flow of | | | | |
| | conversation | | | | |
| 0.62 | N03 Poor rapport | 0.82 | 0.59 | 0.55 | 0.44 |
| 0.48 | G07 Motor | 0.78 | 0.46 | 0.42 | 0.51 |
| | retardation | | | | |
| 0.40 | G16 Active social | 0.77 | 0.34 | 0.39 | 0.26 |
| | avoidance | | | | |
| 40% | Variance explained | 69% | 37% | 31% | 22% |
| | (1-factor model) | | | | |
| 2.8 | Eigenvalue | 4.8 | 2.6 | 2.2 | 1.5 |
| 0.88 | CFI | 0.99 | 0.88 | 0.83 | 0.69 |
| 0.82 | TLI | 0.98 | 0.82 | 0.74 | 0.53 |
| 0.135 | RMSEA | 0.071 | 0.130 | 0.134 | 0.143 |

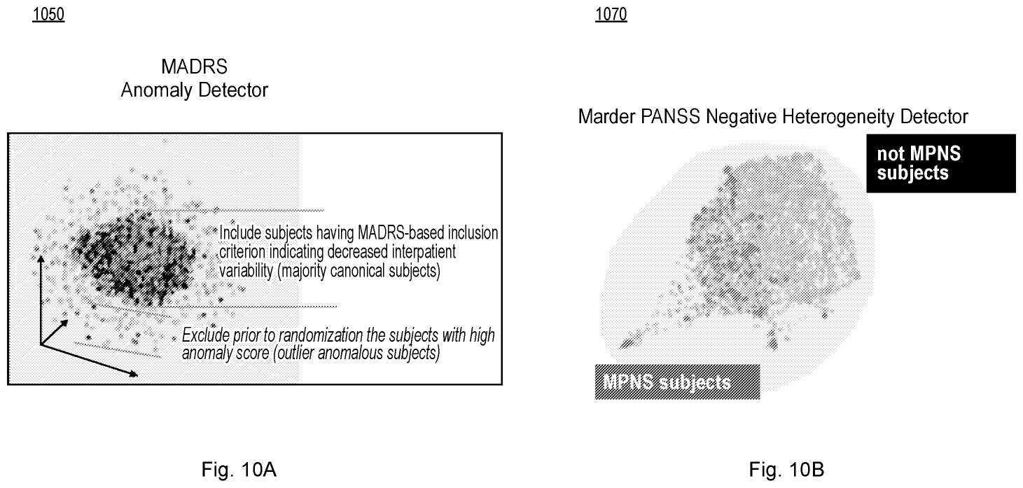

FIG. 10A is a diagram 1050 illustrating an example of the MADRS anomaly detector's inclusion criteria. The subjects shown in darker gray include subjects having MADRS-based inclusion criterion indicating decreased inpatient variability, shown to be clustered in a central group in the X, Y, Z space, and therefore are canonical subjects. However, the subjects shown in lighter gray that are scattered outside of the cluster of MADRS-based included subjects are subjects with high anomaly scores that are excluded prior to randomization.

FIG. 10B is a diagram 1070 illustrating an example of the PANSS Negative Heterogeneity Detector. Subjects in light gray are shown to be not MPNS subjects (e.g., mostly on the right half of the diagram), while subjects in darker gray are MPNS subjects (e.g., mostly on the left half of the diagram).

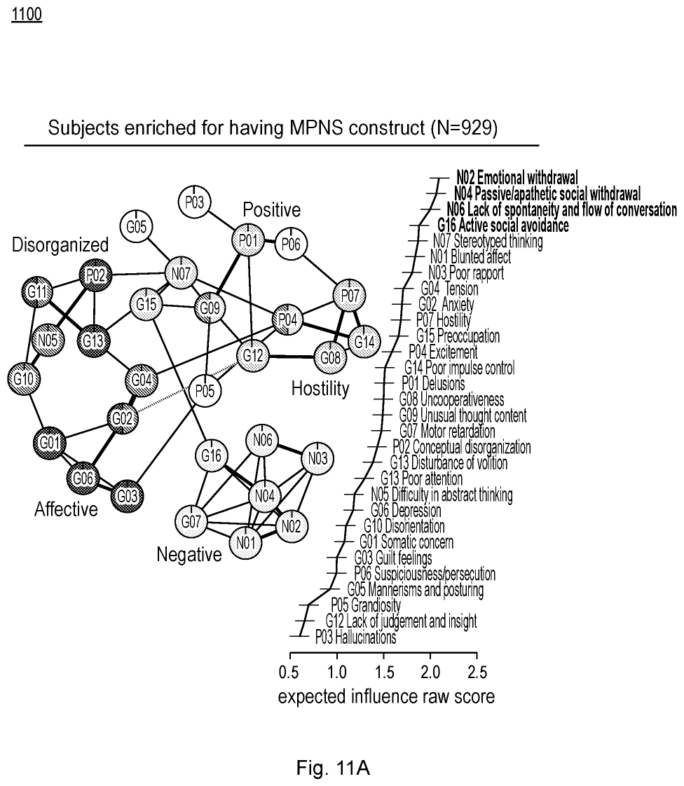

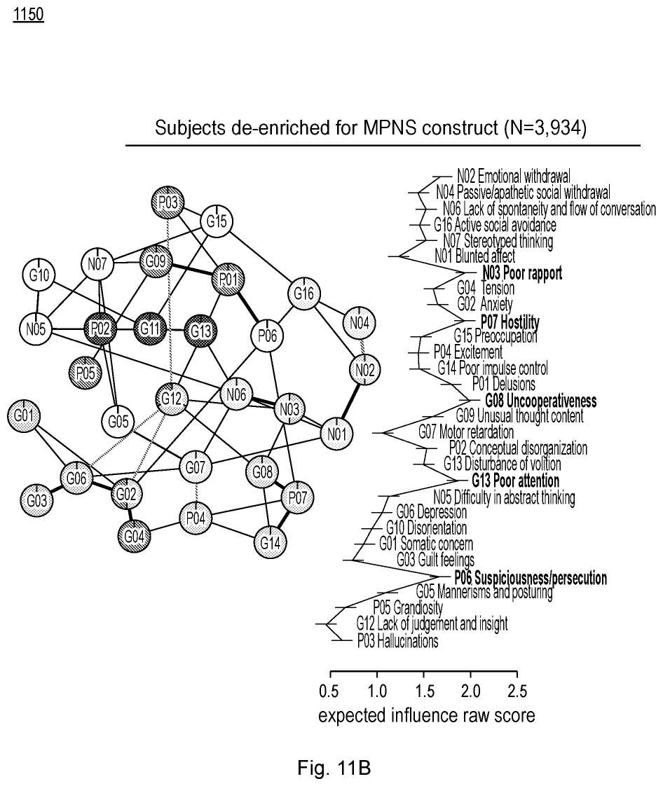

FIG. 11A is a diagram 1100 illustrating a graph of subjects enriched for having MPNS constructs. The graph is based on data having a sample size of 929. With the current method, negative systems N01, N02, N03, N04, N06, and G07 and G16 are isolated in the graph because they are not correlated with the other factors.

FIG. 11B is a diagram 1150 illustrating a graph of subjects de-enriched for having MPNS constructs. In such a graph, the negative symptoms are shown to be throughout the graph and are correlated with positive factors. In contrast, FIG. 11A illustrates that enriching for factor structure enriches a study population better by ensuring the structure, and not severity, is adhered to.

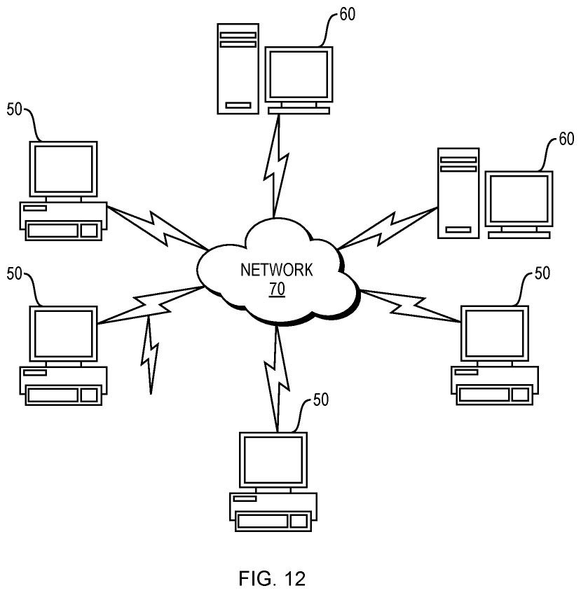

FIG. 12 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

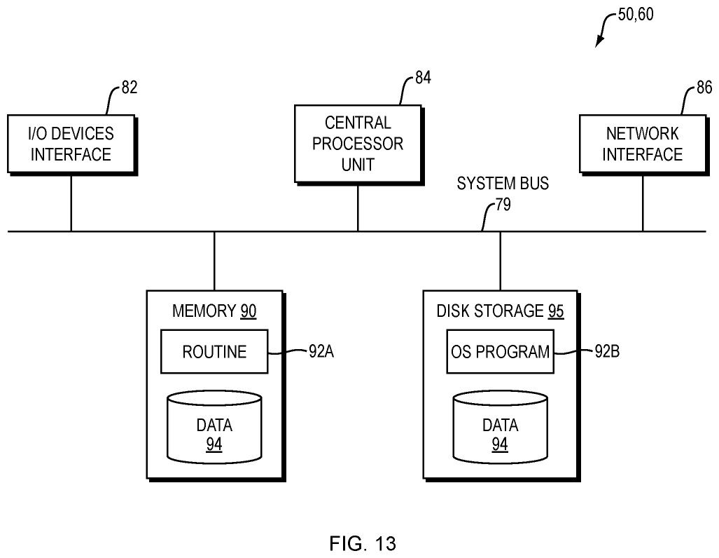

FIG. 13 is a diagram of an example internal structure of a computer (e.g., client processor/device 50 or server computers 60) in the computer system of FIG. 12. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 10). Memory 90 provides volatile storage for computer software instructions 92 and data 94 used to implement some embodiments of the present disclosure (e.g., anomaly detector module, classification module, pre-screening module, vector generation module, and vector analysis module detailed above). Disk storage 95 provides non-volatile storage for computer software instructions 92 and data 94 used to implement some embodiments of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

### Embodiments of the Disclosure

EMBODIMENT 1—A method of verifying eligibility of a subject for a treatment, the method comprising:
representing a plurality of symptoms of the subject in a rating scale as a vector;
computing an anomaly score based on the vector of the subject and a plurality of vectors representing rating scales of other subjects;

based on the anomaly score, ranking the subject with a likelihood of contributing to a subgroup of patients having a common element structure of the rating scale; and

enriching a study population in a clinical trial prior to randomization, the enriched study population having a reduced heterogeneity.

EMBODIMENT 2—The method of Embodiment 1, wherein computing the anomaly score is based on determining a path length from a starting node to an ending node of a binary tree grown from the vector of the subject and the plurality of vectors of the other subjects.

EMBODIMENT 3—The method of Embodiment 2, wherein the starting node is a root node and the ending node is a terminal external node.

EMBODIMENT 4—The method of Embodiment 2, wherein the binary tree is an element of an isolation forest.

EMBODIMENT 5—The method of any of the preceding embodiments, further comprising:
improving a clinical dataset by employing the enriched study population.

EMBODIMENT 6—The method of any of the preceding embodiments, wherein enriching the study population includes verifying a diagnosis of the subject based on the anomaly score.

EMBODIMENT 7—The method of any of the preceding embodiments, wherein enriching the study population includes identifying the subject as an outlier compared to the subgroup of patients based on the anomaly score.

EMBODIMENT 8—The method of any of the preceding embodiments, wherein the plurality of symptoms is of a disease or condition, the method further comprising:
treating the subject having the disease or condition based on the anomaly score.

EMBODIMENT 9—A method of verifying eligibility of a subject for a treatment, the method comprising:
administering a test, to the subject, that measures a plurality of symptoms of the subject;
computing an anomaly score based on a comparison of each element of a plurality of elements of the test administered to a respective expected pattern; and
based on the anomaly score, assigning to the subject a likelihood of having a condition related to the treatment.

EMBODIMENT 10—The method of Embodiment 9, wherein the test is a diagnostic test that measures a subjective condition of the subject.

EMBODIMENT 11—The method of any of Embodiments 9 through 10, wherein the subjective condition is at least one of bipolar disorder, depression, and schizophrenia.

EMBODIMENT 12—The method of Any of Embodiments 9 through 11, wherein the test is at least one of the Montgomery-Åsberg Depression Rating Scale (MADRS) and Positive and Negative Syndrome Scale (PANSS).

EMBODIMENT 13—The method of any of Embodiments 9 through 12, wherein:
administering the test, to the subject, includes administering the test at a plurality of time points; and
computing the anomaly score compares elements at different time points for the subject.

EMBODIMENT 14—The method of any of Embodiments 9 through 13, further comprising:
based on the likelihood of having the condition, determining the subject's eligibility for receiving the treatment.

EMBODIMENT 15—The method of any of Embodiments 9 through 14, further comprising:
based on the likelihood of having the condition, determining the subject's eligibility for a clinical trial of the treatment.

EMBODIMENT 16—The method of any of Embodiments 9 through 15, further comprising:
if the likelihood of the subject having the condition is below a threshold, marking the subject as a candidate for removal from a clinical trial of the treatment; or
otherwise, marking the subject as a candidate for inclusion in a clinical trial of the treatment.

EMBODIMENT 17—The method of any of Embodiments 9 through 16, further comprising:
if the likelihood of the subject having the condition is above a threshold, marking the subject as a candidate for a clinical trial of the treatment.

EMBODIMENT 18—The method of any of Embodiments 9 through 17, wherein computing the anomaly score is performed by weighting the pattern of each element to each other element in accordance to its respective expected pattern.

EMBODIMENT 19—The method of any of Embodiments 9 through 18, further comprising:
computing a plurality of deviance from expected pattern for each element compared to all other elements; and
calculating the anomaly scored based on the plurality of deviance from expected pattern.

EMBODIMENT 20—The method of any of Embodiments 9 through 19, wherein the elements are subjective elements.

EMBODIMENT 21—The method of any of Embodiments 9 through 20, further comprising:
improving a clinical dataset by either including or excluding the subject from the clinical dataset, the inclusion or exclusion based on the assigned likelihood of the subject having the condition.

EMBODIMENT 22—The method of any of Embodiments 9 through 21, further comprising verifying a diagnosis of the subject based on the anomaly score.

EMBODIMENT 23—The method of any of Embodiments 9 through 22, further comprising identifying the subject as an outlier compared to the respective expected pattern based on the anomaly score.

EMBODIMENT 24—The method of any of Embodiments 9 through 23, wherein the plurality of elements is of a disease or condition, the method further comprising:
treating the subject having the disease or condition based on the anomaly score.

EMBODIMENT 25—A method of improving a clinical dataset, the method comprising:
for a subject of the clinical dataset, computing an anomaly score based on a comparison of a plurality of elements of a diagnosis test to respective expected patterns; and
if the anomaly score of the subject is above a particular threshold, removing data corresponding to the subject from the clinical dataset; or
if the anomaly score of the subject is below the particular threshold, including the data corresponding to the subject from the clinical dataset.

EMBODIMENT 26—The method of Embodiment 25, wherein the diagnostic test measures a subjective condition of the subject.

EMBODIMENT 27—The method of any of Embodiments 25 through 26, wherein the subjective condition is at least one of bipolar disorder, depression, and schizophrenia.

EMBODIMENT 28—The method of any of Embodiments 25 through 27, wherein the diagnostic test is at least one of the Montgomery-Åsberg Depression Rating Scale (MADRS) and Positive and Negative Syndrome Scale (PANSS).

EMBODIMENT 29—The method of any of Embodiments 25 through 28, wherein:
administering the diagnostic test, to the subject, includes administering the diagnostic test at a plurality of time points; and
computing the anomaly score compares elements at different time points for the subject.

EMBODIMENT 30—The method of any of Embodiments 25 through 29, further comprising:
based on the likelihood of having a condition that is a prerequisite for a clinical trial, determining eligibility for receiving a treatment or eligibility for a clinical trial.

EMBODIMENT 31—The method of any of Embodiments 25 through 30, further comprising:
if the likelihood of the subject having a condition that is a prerequisite for the clinical trial is below a particular threshold, removing data relating to the subject from the clinical dataset of a clinical trial; or
otherwise, including the data relating to the subject from the clinical dataset of the clinical trial.

EMBODIMENT 32—The method of any of Embodiments 25 through 31, wherein computing the anomaly score is performed by weighting the pattern of each element to each other element in accordance to its respective expected pattern.

EMBODIMENT 33—The method of any of Embodiments 25 through 32, further comprising:
computing a plurality of deviance from expected pattern for each element compared to all other elements; and
calculating the anomaly scored based on the plurality of deviance from expected pattern.

EMBODIMENT 34—The method of any of Embodiments 25 through 33, further comprising, if the anomaly score of the subject is below the particular threshold, verifying a diagnosis of the subject.

EMBODIMENT 35—The method of any of Embodiments 25 through 34, further comprising, if the anomaly score of the subject is above the particular threshold, identifying the subject as an outlier.

EMBODIMENT 36—The method of any of Embodiments 25 through 35, wherein the plurality of elements is of a disease or condition, the method further comprising:
treating the subject having the disease or condition if the anomaly score is below the particular threshold.

EMBODIMENT 37—A method of verifying eligibility of a subject for a treatment of depression, the method comprising:
administering a test, to the subject, that measures a plurality of psychiatric elements of the subject;
computing an anomaly score based on a comparison of each psychiatric element of the plurality of psychiatric elements of the test administered to a respective expected pattern; and
based on the anomaly score, assigning a likelihood of having depression to the subject.

EMBODIMENT 38—The method of Embodiment 37, wherein the test is a diagnostic test that measures depression of the subject.

EMBODIMENT 39—The method of any of Embodiments 37 through 38, wherein the test is at least one of the Montgomery-Åsberg Depression Rating Scale (MADRS) and Positive and Negative Syndrome Scale (PANSS).

EMBODIMENT 40—The method of any of Embodiments 37 through 39, wherein:
administering the test, to the subject, includes administering the test at a plurality of time points; and
computing the anomaly score compares psychiatric elements at different time points for the subject.

EMBODIMENT 41—The method of any of Embodiments 37 through 40, further comprising:
based on the likelihood of having depression, determining eligibility for receiving the treatment.

EMBODIMENT 42—The method of any of Embodiments 37 through 41, further comprising:
based on the likelihood of having depression, determining eligibility for a clinical trial of the treatment.

EMBODIMENT 43—The method of any of Embodiments 37 through 42, further comprising:
if the likelihood of the subject having depression is below a threshold, removing data relating to the subject from a dataset of a clinical trial of the treatment; and
otherwise, including the data relating to the subject from the dataset of the clinical trial of the treatment.

EMBODIMENT 44—The method of any of Embodiments 37 through 43, wherein computing the anomaly score is performed by weighting the pattern of each psychiatric element to each other psychiatric element in accordance to its respective expected pattern.

EMBODIMENT 45—The method of any of Embodiments 37 through 44, further comprising:
computing a plurality of deviance from expected pattern for each psychiatric element compared to all other psychiatric elements; and
calculating the anomaly scored based on the plurality of deviance from expected pattern.

EMBODIMENT 46—The method of any of Embodiments 37 through 45, further comprising:
improving a clinical dataset by including or excluding the subject using the likelihood of having depression.

EMBODIMENT 47—The method of any of Embodiments 37 through 46, further comprising:
verifying a diagnosis of the subject based on the anomaly score.

EMBODIMENT 48—The method of any of Embodiments 37 through 47, further comprising:
identifying the subject as an outlier compared to the subgroup of patients based on the anomaly score.

EMBODIMENT 49—The method of any of Embodiments 37 through 48, further comprising:
treating the subject having a disease or condition of depression based on the anomaly score.

EMBODIMENT 50—A method of verifying a diagnosis of a subject, the method comprising:
administering a test, to the subject, that measures a plurality of elements of the subject;
computing an anomaly score based on a comparison of each element of the plurality of elements of the test administered to a respective expected pattern; and
based on the anomaly score, assigning a likelihood of having a condition related to the treatment to the subject.

EMBODIMENT 51—The method of Embodiment 50, wherein the test is a diagnostic test that measures a subjective condition of the subject.

EMBODIMENT 52—The method of any of Embodiments 50 through 51, wherein the subjective condition is at least one of bipolar disorder, depression, and schizophrenia.

EMBODIMENT 53—The method of any of Embodiments 50 through 52, wherein the test is at least one of the Montgomery-Åsberg Depression Rating Scale (MADRS) and Positive and Negative Syndrome Scale (PANSS).

EMBODIMENT 54—The method of any of Embodiments 50 through 53, wherein:
administering the test, to the subject, includes administering the test at a plurality of time points; and
computing the anomaly score compares elements at different time points for the subject.

EMBODIMENT 55—The method of any of Embodiments 50 through 54, further comprising:
based on the likelihood of having the condition, verifying the diagnosis.

EMBODIMENT 56—The method of any of Embodiments 50 through 55, further comprising:
based on the likelihood of having the condition, determining eligibility for a clinical trial related to the diagnosis.

EMBODIMENT 57—The method of any of Embodiments 50 through 56, further comprising:
if the likelihood of the subject having the condition is below a particular threshold, removing data relating to the subject from a dataset of a clinical trial of the treatment; or
otherwise, including the data relating to the subject from the dataset of the clinical trial of the treatment.

EMBODIMENT 58—The method of any of Embodiments 50 through 57, wherein computing the anomaly score is performed by weighting the pattern of each element to each other element in accordance to its respective expected pattern.

EMBODIMENT 59—The method of any of Embodiments 50 through 58, further comprising:
computing a plurality of deviance from expected pattern for each element compared to all other elements; and
calculating the anomaly scored based on the plurality of deviance from expected pattern.

EMBODIMENT 60—The method of any of Embodiments 50 through 59, further comprising:
improving a clinical dataset by excluding the subject if the likelihood of having the condition is below a particular threshold.

EMBODIMENT 61—The method of any of Embodiments 50 through 60, further comprising verifying a diagnosis of the subject based on the anomaly score.

EMBODIMENT 62—The method of any of Embodiments 50 through 61, further comprising identifying the subject as an outlier based on the anomaly score.

EMBODIMENT 63—The method of any of Embodiments 50 through 62, further comprising:
treating the subject having the condition based on the anomaly score.

EMBODIMENT 64—A method of treating a subject having a psychiatric condition, the method comprising:
administering to the subject a therapeutically effective amount of a treatment for the condition,
wherein, prior to receiving the treatment, the subject was determined to be eligible for the treatment by:
administering a test, to the subject, that measures a plurality of elements of the subject;
computing an anomaly score based on a comparison of each element of the plurality of elements of the test administered to a respective expected pattern; and
based on the anomaly score, assigning to the subject a likelihood of having the psychiatric condition.

EMBODIMENT 65—The method of Embodiment 64, wherein the treatment is administered to the subject in a clinical trial.

EMBODIMENT 66—The method of any of Embodiments 64 through 65, wherein the psychiatric condition includes depressive episodes.

EMBODIMENT 67—The method of any of Embodiments 64 through 66, wherein the psychiatric condition is bipolar I depression.

EMBODIMENT 68—The method of any of Embodiments 64 through 67, wherein the test that measures a plurality of elements of the subject is the Montgomery-Åsberg Depression Rating Scale (MADRS) test.

EMBODIMENT 69—The method of any of Embodiments 64 through 68, wherein the treatment includes administration of a therapeutic agent.

EMBODIMENT 70—The method of any of Embodiments 64 through 69, further comprising:
improving a clinical dataset by excluding the subject if the anomaly score is above a particular threshold.

EMBODIMENT 71—The method of any of Embodiments 64 through 70, further comprising verifying a diagnosis of the subject based on the anomaly score.

EMBODIMENT 72—The method of any of Embodiments 64 through 71, further comprising identifying the subject as an outlier based on the anomaly score.

EMBODIMENT 73—The method of any of Embodiments 64 through 72, further comprising:
treating the subject having the condition based on the anomaly score.

EMBODIMENT 74—A method of treating a subject having bipolar I depression, the method comprising:
administering to the subject a therapeutically effective amount of a therapeutic agent,
wherein, prior to receiving the therapeutically effective amount, the subject was determined to be eligible for a treatment comprising the therapeutic agent by:
administering a Montgomery-Åsberg Depression Rating Scale (MADRS) test, to the subject, that measures a plurality of elements of the subject;
computing an anomaly score based on a comparison of each element of the plurality of elements of the MADRS test administered to a respective expected pattern; and
based on the anomaly score, assigning a likelihood of having bipolar I depression to the subject.

EMBODIMENT 75—The method of Embodiment 74, further comprising:
improving a clinical dataset by excluding the subject if the anomaly score is above a particular threshold.

EMBODIMENT 76—The method of any of Embodiments 74 through 75, further comprising verifying a diagnosis of bipolar I depression of the subject based on the anomaly score.

EMBODIMENT 77—The method of any of Embodiments 74 through 76, further comprising identifying the subject as an outlier based on the anomaly score.

EMBODIMENT 78—The method of any of Embodiments 74 through 77, further comprising:
treating the subject having bipolar I depression based on the anomaly score.

EMBODIMENT 79—A method of verifying treatment eligibility of a subject exhibiting a plurality of symptoms characterized in a rating scale, the method comprising:
characterizing the subject's plurality of symptoms in the rating scale as a subject vector;
characterizing a plurality of other subjects' plurality of symptoms in the rating scale as a plurality of population vectors, each population vector corresponding with one of the plurality of other subjects; and
computing an anomaly score based on the subject vector and the plurality of population vectors; and
based on the anomaly score, verifying treatment eligibility of the subject by ranking the subject with a likelihood of contributing to a sub-population of subjects having a common element structure of the rating scale.

EMBODIMENT 80—The method of Embodiment 79, further comprising:
improving a clinical dataset by excluding the subject if the anomaly score is above a particular threshold.

EMBODIMENT 81—The method of any of Embodiments 79 through 80, wherein verifying treatment eligibility further includes verifying a diagnosis of the subject based on the anomaly score.

EMBODIMENT 82—The method of any of Embodiments 79 through 81, further comprising identifying the subject as an outlier based on the anomaly score.

EMBODIMENT 83—The method of any of Embodiments 79 through 82, further comprising:
treating the subject having the condition based on the anomaly score.

EMBODIMENT 84—A method for determining subject participation in a clinical trial comprising:
receiving one or more rater inputs reflecting the rater's clinical evaluation of a severity of a previously diagnosed condition in a subject; and
performing a computerized assessment of the subject to quantify severity of the previously diagnosed condition in the subject through a computerized interview that comprises:
presenting a plurality of questions to the subject and receiving a plurality of corresponding inputs from the subject in response thereto;
based on plurality of inputs received from the subject, determining an anomaly score for the condition in the subject; and
determining, via a processor, a recommendation of including or excluding the subject from the clinical trial.

EMBODIMENT 85—A computer-implemented method of identifying one or more clinical study candidates, the method comprises:
receiving consolidated health care information for a consumer of medical services;
retrieving, by one or more computers, attributes defining a suitable candidate for a clinical study, the attributes based on rating data of patients tested for a condition;
causing the one or more computers to compare the attributes defining the suitable candidate for the clinical study to the consolidated health information for the consumer;
determining by the one or more computers that the consumer's consolidated health information includes at least one of the attributes defining the suitable candidate for the clinical study;
identifying the consumer as eligible to participate in the clinical study; and
notifying an administrator of the clinical study that the consumer is eligible to participate in the clinical study.

EMBODIMENT 86—A method of enriching a study population in a clinical trial, the method comprising:
representing a plurality of symptoms of a subject in a rating scale as a vector;
computing a score based on the vector of the subject and a plurality of vectors representing rating scales of other subjects; and

based on the score, ranking the subject with a likelihood of contributing to a subgroup of patients having a common element structure of the rating scale, the ranking based on variance explained by a set of negative symptoms of the plurality of symptoms.

EMBODIMENT 87—The method of Embodiment 86, further comprising:
improving a clinical dataset by employing the enriched study population.

EMBODIMENT 88—The method of any one of Embodiments 86 through 87, wherein enriching the study population includes verifying a diagnosis of the subject based on the score.

EMBODIMENT 89—The method of any one of Embodiments 86 through 88, wherein enriching the study population includes identifying the subject as an outlier compared to the subgroup of patients based on the score.

EMBODIMENT 90—The method of any one of Embodiments 86 through 89, wherein the plurality of symptoms is of a disease or condition, the method further comprising:
treating the subject having the disease or condition based on the score.

EMBODIMENT 91—The method of Embodiment 90, wherein the subject's plurality of symptoms in a rating scale is presented as the Montgomery-Åsberg Depression Rating Scale (MADRS) or the Positive and Negative Syndrome Scale (PANSS).

EMBODIMENT 92—The method of any one of Embodiments 86-91, further comprising:
representing the subject's plurality of symptoms in a rating scale at a plurality of time points; and
computing the score compares elements at different time points for the subject.

EMBODIMENT 93—The method of Embodiment 92, further comprising:
based on a likelihood of having a disease or condition as determined by the rating scale, determining the subject's eligibility for receiving the treatment.

EMBODIMENT 94—The method of any one of Embodiments 91 through 92, further comprising:
based on a likelihood of having a disease or condition as determined by the rating scale, determining the subject's eligibility for a clinical trial of the treatment.

EMBODIMENT 95—The method of any one of Embodiments 86-94, wherein the likelihood of contributing to the subgroup of patients is based on, at least in part, having a disease or condition relevant to the clinical trial.

EMBODIMENT 96—A method of enriching a study population in a clinical trial, the method comprising:
computing a score based on a vector representing a plurality of symptoms of a subject in a rating scale, and a plurality of vectors representing rating scales of other subjects; and

based on the score, ranking the subject with a likelihood of contributing to a subgroup of patients having a common element structure of the rating scale.

EMBODIMENT 97—A method of enriching a study population in a clinical trial, the method comprising:
computing a plurality of scores based on a plurality of vectors, each vector representing a plurality of symptoms of a subject in a rating scale; and

based on the plurality of scores, ranking each subject with a likelihood of contributing to a subgroup of patients having a common element structure of the rating scale.

What is claimed is:

1. A method of verifying eligibility of a subject for a treatment, the method comprising:
representing a plurality of symptoms of the subject in a rating scale as a vector;
computing an anomaly score based on the vector of the subject and a plurality of vectors representing rating scales of other subjects;
based on the anomaly score, ranking the subject with a likelihood of contributing to a subgroup of patients having a common element structure of the rating scale; and
enriching a study population in a clinical trial prior to randomization, the enriched study population having a reduced heterogeneity.

2. The method of claim 1, wherein computing the anomaly score is based on determining a path length from a starting node to an ending node of a binary tree grown from the vector of the subject and the plurality of vectors of the other subjects.

3. The method of claim 2, wherein the starting node is a root node and the ending node is a terminal external node.

4. The method of claim 2, wherein the binary tree is an element of an isolation forest.

5. The method of claim 1, further comprising:
improving a clinical dataset by employing the enriched study population.

6. The method of claim 1, wherein enriching the study population includes verifying a diagnosis of the subject based on the anomaly score.

7. The method of claim 1, wherein enriching the study population includes identifying the subject as an outlier compared to the subgroup of patients based on the anomaly score.

8. The method of claim 1, wherein the plurality of symptoms is of a disease or condition, the method further comprising:
treating the subject having the disease or condition based on the anomaly score.

9. A method of verifying eligibility of a subject for a treatment, the method comprising:
administering a test, to the subject, that measures a plurality of symptoms of the subject;
computing an anomaly score based on a comparison of each element of a plurality of elements of the test administered to a respective expected pattern; and
based on the anomaly score, assigning to the subject a likelihood of having a condition related to the treatment.

10. The method of claim 9, wherein the test is a diagnostic test that measures a subjective condition of the subject.

11. The method of claim 10, wherein the subjective condition is at least one of bipolar disorder, depression, and schizophrenia.

12. The method of claim 9, wherein the test is at least one of the Montgomery-Åsberg Depression Rating Scale (MADRS) and Positive and Negative Syndrome Scale (PANSS).

13. The method of claim 9, wherein:
administering the test, to the subject, includes administering the test at a plurality of time points; and
computing the anomaly score compares elements at different time points for the subject.

14. The method of claim 9, further comprising:
based on the likelihood of having the condition, determining the subject's eligibility for receiving the treatment.

15. The method of claim 9, further comprising:
based on the likelihood of having the condition, determining the subject's eligibility for a clinical trial of the treatment.

16. The method of claim 9, further comprising:
if the likelihood of the subject having the condition is below a threshold, marking the subject as a candidate for removal from a clinical trial of the treatment; or
otherwise, marking the subject as a candidate for inclusion in a clinical trial of the treatment.

17. The method of claim 9, further comprising:
if the likelihood of the subject having the condition is above a threshold, marking the subject as a candidate for a clinical trial of the treatment.

18. The method of claim 9, wherein computing the anomaly score is performed by weighting the pattern of each element to each other element in accordance to its respective expected pattern.

19. The method of claim 9, further comprising:
computing a plurality of deviance from expected pattern for each element compared to all other elements; and
calculating the anomaly scored based on the plurality of deviance from expected pattern.

20. The method of claim 9, wherein the elements are subjective elements.

21. The method of claim 9, further comprising:
improving a clinical dataset by either including or excluding the subject from the clinical dataset, the inclusion or exclusion based on the assigned likelihood of the subject having the condition.

22. The method of claim 9, further comprising verifying a diagnosis of the subject based on the anomaly score.

23. The method of claim 9, further comprising identifying the subject as an outlier compared to the respective expected pattern based on the anomaly score.

24. The method of claim 9, wherein the plurality of elements is of a disease or condition, the method further comprising:
treating the subject having the disease or condition based on the anomaly score.

25. A method of improving a clinical dataset, the method comprising:
for a subject of the clinical dataset, computing an anomaly score based on a comparison of a plurality of elements of a diagnosis test to respective expected patterns; and
if the anomaly score of the subject is above a particular threshold, removing data corresponding to the subject from the clinical dataset; or
if the anomaly score of the subject is below the particular threshold, including the data corresponding to the subject from the clinical dataset.

26. The method of claim 25, wherein the diagnostic test measures a subjective condition of the subject.

27. The method of claim 26, wherein the subjective condition is at least one of bipolar disorder, depression, and schizophrenia.

28. The method of claim 25, wherein the diagnostic test is at least one of the Montgomery-Åsberg Depression Rating Scale (MADRS) and Positive and Negative Syndrome Scale (PANSS).

29. The method of claim 25, wherein:
administering the diagnostic test, to the subject, includes administering the diagnostic test at a plurality of time points; and
computing the anomaly score compares elements at different time points for the subject.

30. The method of claim 25, further comprising:
based on the likelihood of having a condition that is a prerequisite for a clinical trial, determining eligibility for receiving a treatment or eligibility for a clinical trial.

31. The method of claim 25, further comprising:
if the likelihood of the subject having a condition that is a prerequisite for the clinical trial is below a particular threshold, removing data relating to the subject from the clinical dataset of a clinical trial; or
otherwise, including the data relating to the subject from the clinical dataset of the clinical trial.

32. The method of claim 25, wherein computing the anomaly score is performed by weighting the pattern of each element to each other element in accordance to its respective expected pattern.

33. The method of claim 25, further comprising:
computing a plurality of deviance from expected pattern for each element compared to all other elements; and
calculating the anomaly scored based on the plurality of deviance from expected pattern.

34. The method of claim 25, further comprising, if the anomaly score of the subject is below the particular threshold, verifying a diagnosis of the subject.

35. The method of claim 25, further comprising, if the anomaly score of the subject is above the particular threshold, identifying the subject as an outlier.

36. The method of claim 25, wherein the plurality of elements is of a disease or condition, the method further comprising:
treating the subject having the disease or condition if the anomaly score is below the particular threshold.

37. A method of verifying eligibility of a subject for a treatment of depression, the method comprising:
administering a test, to the subject, that measures a plurality of psychiatric elements of the subject;
computing an anomaly score based on a comparison of each psychiatric element of the plurality of psychiatric elements of the test administered to a respective expected pattern; and
based on the anomaly score, assigning a likelihood of having depression to the subject.

38. The method of claim 37, wherein the test is a diagnostic test that measures depression of the subject.

39. The method of claim 37, wherein the test is at least one of the Montgomery-Åsberg Depression Rating Scale (MADRS) and Positive and Negative Syndrome Scale (PANSS).

40. The method of claim 37, wherein:
administering the test, to the subject, includes administering the test at a plurality of time points; and
computing the anomaly score compares psychiatric elements at different time points for the subject.

41. The method of claim 37, further comprising:
based on the likelihood of having depression, determining eligibility for receiving the treatment.

42. The method of claim 37, further comprising:
based on the likelihood of having depression, determining eligibility for a clinical trial of the treatment.

43. The method of claim 37, further comprising:
if the likelihood of the subject having depression is below a threshold, removing data relating to the subject from a dataset of a clinical trial of the treatment; and
otherwise, including the data relating to the subject from the dataset of the clinical trial of the treatment.

44. The method of claim 37, wherein computing the anomaly score is performed by weighting the pattern of each psychiatric element to each other psychiatric element in accordance to its respective expected pattern.

45. The method of claim 37, further comprising:
computing a plurality of deviance from expected pattern for each psychiatric element compared to all other psychiatric elements; and
calculating the anomaly scored based on the plurality of deviance from expected pattern.

46. The method of claim 37, further comprising:
improving a clinical dataset by including or excluding the subject using the likelihood of having depression.

47. The method of claim 37, further comprising:
verifying a diagnosis of the subject based on the anomaly score.

48. The method of claim 37, further comprising:
identifying the subject as an outlier compared to the subgroup of patients based on the anomaly score.

49. The method of claim 37, further comprising:
treating the subject having a disease or condition of depression based on the anomaly score.

50. A method of verifying a diagnosis of a subject, the method comprising:
administering a test, to the subject, that measures a plurality of elements of the subject;
computing an anomaly score based on a comparison of each element of the plurality of elements of the test administered to a respective expected pattern; and
based on the anomaly score, assigning a likelihood of having a condition related to the treatment to the subject.

51. The method of claim 50, wherein the test is a diagnostic test that measures a subjective condition of the subject.

52. The method of claim 51, wherein the subjective condition is at least one of bipolar disorder, depression, and schizophrenia.

53. The method of claim 50, wherein the test is at least one of the Montgomery-Åsberg Depression Rating Scale (MADRS) and Positive and Negative Syndrome Scale (PANSS).

54. The method of claim 50, wherein:
administering the test, to the subject, includes administering the test at a plurality of time points; and
computing the anomaly score compares elements at different time points for the subject.

55. The method of claim 50, further comprising:
based on the likelihood of having the condition, verifying the diagnosis.

56. The method of claim 50, further comprising:
based on the likelihood of having the condition, determining eligibility for a clinical trial related to the diagnosis.

57. The method of claim 50, further comprising:
if the likelihood of the subject having the condition is below a particular threshold, removing data relating to the subject from a dataset of a clinical trial of the treatment; or
otherwise, including the data relating to the subject from the dataset of the clinical trial of the treatment.

58. The method of claim 50, wherein computing the anomaly score is performed by weighting the pattern of each element to each other element in accordance to its respective expected pattern.

59. The method of claim 50, further comprising:
computing a plurality of deviance from expected pattern for each element compared to all other elements; and
calculating the anomaly scored based on the plurality of deviance from expected pattern.

60. The method of claim 50, further comprising:
improving a clinical dataset by excluding the subject if the likelihood of having the condition is below a particular threshold.

61. The method of claim 50, further comprising verifying a diagnosis of the subject based on the anomaly score.

62. The method of claim 50, further comprising identifying the subject as an outlier based on the anomaly score.

63. The method of claim 50, further comprising:
treating the subject having the condition based on the anomaly score.

64. A method of treating a subject having a psychiatric condition, the method comprising:
administering to the subject a therapeutically effective amount of a treatment for the condition,
wherein, prior to receiving the treatment, the subject was determined to be eligible for the treatment by:
administering a test, to the subject, that measures a plurality of elements of the subject;
computing an anomaly score based on a comparison of each element of the plurality of elements of the test administered to a respective expected pattern; and
based on the anomaly score, assigning to the subject a likelihood of having the psychiatric condition.

65. The method of claim 64, wherein the treatment is administered to the subject in a clinical trial.

66. The method of claim 64, wherein the psychiatric condition includes depressive episodes.

67. The method of claim 64, wherein the psychiatric condition is bipolar I depression.

68. The method of claim 64, wherein the test that measures a plurality of elements of the subject is the Montgomery-Åsberg Depression Rating Scale (MADRS) test.

69. The method of claim 64, wherein the treatment includes administration of a therapeutic agent.

70. The method of claim 64, further comprising:
improving a clinical dataset by excluding the subject if the anomaly score is above a particular threshold.

71. The method of claim 64, further comprising verifying a diagnosis of the subject based on the anomaly score.

72. The method of claim 64, further comprising identifying the subject as an outlier based on the anomaly score.

73. The method of claim 64, further comprising:
treating the subject having the condition based on the anomaly score.

74. A method of treating a subject having bipolar I depression, the method comprising:
administering to the subject a therapeutically effective amount of a therapeutic agent,
wherein, prior to receiving the therapeutically effective amount, the subject was determined to be eligible for a treatment comprising the therapeutic agent by:
administering a Montgomery-Åsberg Depression Rating Scale (MADRS) test, to the subject, that measures a plurality of elements of the subject;
computing an anomaly score based on a comparison of each element of the plurality of elements of the MADRS test administered to a respective expected pattern; and
based on the anomaly score, assigning a likelihood of having bipolar I depression to the subject.

75. The method of claim 74, further comprising:
improving a clinical dataset by excluding the subject if the anomaly score is above a particular threshold.

76. The method of claim 74, further comprising verifying a diagnosis of bipolar I depression of the subject based on the anomaly score.

77. The method of claim 74, further comprising identifying the subject as an outlier based on the anomaly score.

78. The method of claim 74, further comprising:
treating the subject having bipolar I depression based on the anomaly score.

79. A method of verifying treatment eligibility of a subject exhibiting a plurality of symptoms characterized in a rating scale, the method comprising:
characterizing the subject's plurality of symptoms in the rating scale as a subject vector;
characterizing a plurality of other subjects' plurality of symptoms in the rating scale as a plurality of population vectors, each population vector corresponding with one of the plurality of other subjects; and
computing an anomaly score based on the subject vector and the plurality of population vectors; and
based on the anomaly score, verifying treatment eligibility of the subject by ranking the subject with a likelihood of contributing to a sub-population of subjects having a common element structure of the rating scale.

80. The method of claim 79, further comprising:
improving a clinical dataset by excluding the subject if the anomaly score is above a particular threshold.

81. The method of claim 79, wherein verifying treatment eligibility further includes verifying a diagnosis of the subject based on the anomaly score.

82. The method of claim 79, further comprising identifying the subject as an outlier based on the anomaly score.

83. The method of claim 79, further comprising:
treating the subject having the condition based on the anomaly score.

84. A method for determining subject participation in a clinical trial comprising:
receiving one or more rater inputs reflecting the rater's clinical evaluation of a severity of a previously diagnosed condition in a subject; and
performing a computerized assessment of the subject to quantify severity of the previously diagnosed condition in the subject through a computerized interview that comprises:
presenting a plurality of questions to the subject and receiving a plurality of corresponding inputs from the subject in response thereto;
based on plurality of inputs received from the subject, determining an anomaly score for the condition in the subject; and
determining, via a processor, a recommendation of including or excluding the subject from the clinical trial.

85. A computer-implemented method of identifying one or more clinical study candidates, the method comprises:
receiving consolidated health care information for a consumer of medical services;
retrieving, by one or more computers, attributes defining a suitable candidate for a clinical study, the attributes based on rating data of patients tested for a condition;
causing the one or more computers to compare the attributes defining the suitable candidate for the clinical study to the consolidated health information for the consumer;
determining by the one or more computers that the consumer's consolidated health information includes at least one of the attributes defining the suitable candidate for the clinical study;
identifying the consumer as eligible to participate in the clinical study; and
notifying an administrator of the clinical study that the consumer is eligible to participate in the clinical study.

86. A method of enriching a study population in a clinical trial, the method comprising:
representing a plurality of symptoms of a subject in a rating scale as a vector;
computing a score based on the vector of the subject and a plurality of vectors representing rating scales of other subjects; and
based on the score, ranking the subject with a likelihood of contributing to a subgroup of patients having a common element structure of the rating scale, the ranking based on variance explained by a set of negative symptoms of the plurality of symptoms.

87. The method of claim 86, further comprising:
improving a clinical dataset by employing the enriched study population.

88. The method of any one of claims 86-87, wherein enriching the study population includes verifying a diagnosis of the subject based on the score.

89. The method of any one of claims 86-88, wherein enriching the study population includes identifying the subject as an outlier compared to the subgroup of patients based on the score.

90. The method of any one of claims 86-89, wherein the plurality of symptoms is of a disease or condition, the method further comprising:
treating the subject having the disease or condition based on the score.

91. The method of claim 90, wherein the subject's plurality of symptoms in a rating scale is presented as the Montgomery-Åsberg Depression Rating Scale (MADRS) or the Positive and Negative Syndrome Scale (PANSS).

92. The method of any one of claims 86-91, further comprising:
representing the subject's plurality of symptoms in a rating scale at a plurality of time points; and
computing the score compares elements at different time points for the subject.

93. The method of claim 92, further comprising:
based on a likelihood of having a disease or condition as determined by the rating scale, determining the subject's eligibility for receiving the treatment.

94. The method of claim 92, further comprising:
based on a likelihood of having a disease or condition as determined by the rating scale, determining the subject's eligibility for a clinical trial of the treatment.

95. The method of any one of claims 86-94, wherein the likelihood of contributing to the subgroup of patients is based on, at least in part, having a disease or condition relevant to the clinical trial.

96. A method of enriching a study population in a clinical trial, the method comprising:
computing a score based on a vector representing a plurality of symptoms of a subject in a rating scale, and a plurality of vectors representing rating scales of other subjects; and
based on the score, ranking the subject with a likelihood of contributing to a subgroup of patients having a common element structure of the rating scale.

97. A method of enriching a study population in a clinical trial, the method comprising:
computing a plurality of scores based on a plurality of vectors, each vector representing a plurality of symptoms of a subject in a rating scale; and
based on the plurality of scores, ranking each subject with a likelihood of contributing to a subgroup of patients having a common element structure of the rating scale.
